(19) United States
(12) Patent Application Publication
(10) Pub. No.: US 2018/0110849 A1
(43) Pub. Date: Apr. 26, 2018

(54) METHODS FOR IMMUNIZING AGAINST CLOSTRIDIUM DIFFICILE

(71) Applicant: Sanofi Pasteur, Inc., Swiftwater, PA (US)

(72) Inventors: Laurence Quemeneur, Lyon Cedex 07 (FR); Patricia Pietrobon, Swiftwater, PA (US); Patricia Ladono-Hayes, Lyon Cedex 07 (FR); Steven HAUSER, Swiftwater, PA (US)

(73) Assignee: Sanofi Pasteur, Inc., Swiftwater, PA (US)

(21) Appl. No.: 15/573,351

(22) PCT Filed: May 14, 2016
(86) PCT No.: PCT/US2016/032568

§ 371 Date: Nov. 10, 2017

Related U.S. Application Data
(60) Provisional application No. 62/162,357, filed on May 15, 2015.

(51) Int. Cl. A61K 39/08

(52) U.S. Cl. CPC A61K 39/08; A61K 2039/575

(57) ABSTRACT

The disclosure relates to generally to the field of therapeutic and/or protective vaccination against Clostridium dificile (C. difficile). More specifically, it relates to methods for immunizing a host against C. difficile strains expressing C. difficile binary toxin (CDT) and strains not expressing CDT. These methods involve the administration to a host of an immunogenic composition comprising inactivated purified C. difficile Toxin A and purified Toxin B. The purified C. difficile toxins may be derived from a C. difficile strain that does not express CDT.

### RELATED APPLICATIONS

This application claims priority to U.S. Appln. No. 62/162,357 filed May 15, 2015.

### FIELD OF THE DISCLOSURE

The disclosure relates generally to the field of therapeutic and/or protective vaccination against Clostridium difficile (C. difficile).

### BACKGROUND OF THE DISCLOSURE

C. difficile is a widely distributed pathogen with multiple strain toxinotypes/PCR-ribotypes (RT). A number of molecular epidemiology studies conducted across several countries have been published over the last five years. Results demonstrated that circulating strains encoding several toxin variants are prevalent as the cause of human symptomatic C. difficile infection (CDI). The five most prevalent include toxinotypes 0, III, IV, V and VIII. There is a need in the art for compositions including C. difficile antigens for use as therapeutic and/or protective immunogenic compositions (e.g., vaccines) against multiple C. difficile strains/toxinotypes, especially against both those that express C. difficile binary toxin (“CDT”; e.g., from the CDTa and/or CDTb subunits) and those that do not, especially where the vaccine is prepared from a strain that does not express CDT and the strain for which immunization is desired does express CDT. It has been recognized in the art, for instance, that recombinant Toxin A and Toxin B may not provide protection against strains expressing CDT unless binary toxin is included in the vaccine (WO 2013/112867). Example 11 of the '867 application explains that “recombinant TcdA+TcdB vaccine was unable to substantially increase the survival of hamsters challenged with . . . spores . . . from a highly virulent NAP1/027/BI17 strain” (which expresses CDT) but that “the addition of binary toxin (CDTa and CDTb) to the vaccine . . . consistently increased the protective efficacy of this vaccine”. And, in fact, only the inclusion of “both binary toxin proteins (CDTa and CDTb) with TcdA and TcdB fully restored . . . protection.” Contrary to these teachings, and surprisingly in view of the same, such problems have been overcome using the reagents and methods described in this disclosure.

### BRIEF DESCRIPTION OF THE DRAWINGS

FIG. 1 is a graphical representation of the results of a cytotoxicity assay. A cytotoxicity assay using IMR90 cells was conducted using samples from one batch of each of toxin A and toxin B that underwent inactivation in accordance to the described methods (Example 2). Samples were taken on day 0, following addition of formaldehyde to inactivate the toxin and on a number of days later to assess the cytotoxicity of the material. The y-axis identifies the minimum concentration at which 50% of the cells became rounded (as opposed to their normal striated morphology) in the presence of toxic material (MC50). The lower limit of detection value (LOD) for the assay is identified using a dashed line.

FIG. 2 is a schematic representation of an exemplary method of inactivating C. difficile Toxin A and Toxin B.

FIG. 3 is a graphical representation of the results from an immunization study. In the study (described in Example 2) conducted in hamster challenge model (using 5 groups with 9 hamsters/group), Toxoid A and Toxoid B were prepared in accordance to the described methods, combined and formulated as a lyophilized composition. The composition was reconstituted and adjuvanted prior to vaccination. One hamster group was administered a placebo. Four different dilutions of a human dose (HD) of the composition (100 pg/dose) were prepared, one for each of the four other hamster groups. Compositions administered (i.e., placebo or HD dilution) are identified on X-axis. The % survival of each group (Y-axis) following administration of a lethal challenge dose of C. difficile was determined as is graphically shown.

FIGS. 4A-B illustrates cytotoxicity assay results (using IMR-90 cells) for a number of representative toxinotype clinical isolates as described in Example 3. In panel A, calculated IC50 results are plotted against the concentration of Toxin A present in bacterial supernatant (μg/ml). In panel B, calculated IC50 results are plotted against the concentration of Toxin B present in bacterial supernatant (μg/ml).

FIG. 5 provides cross-neutralization assay results as described in Example 3. The relative efficacy (RE) of vaccine specific antibodies to neutralie the respective cytotoxic activity of bacterial supernatant from various toxinotype representative clinical isolates was assayed. Depicted is the RE of each isolate against their respective cytotoxic index. The calculated cross-seroneutralization significance threshold RE was 5.4, based on results obtained with hyperimmune irrevalent hamster serum and heterlogous strains.

FIGS. 6A-B illustrates results of a hamster challenge study as described in Example 4 using the C. difficile vaccine strain as the challenge strain. Shown in panel A is feces/scoring (diarrheal disease) over time in the vaccine group (^{▪}) as compared to the placebo control group (^{∘}). Shown in panel B is percent survival overtime in the vaccine group (^{▪}) as compared to the placebo control group (^{∘}).

FIGS. 7A-B provide results of a hamster challenge study as described in Example 4 using a Toxinotype 0 PCR-ribotype 012 strain as the challenge strain. Shown in panel A is feces/scoring (diarrheal disease) over time in the vaccine group (^{▪}) as compared to the placebo control group (^{∘}). Shown in panel B is percent survival overtime in the vaccine group as compared to the placebo control group.

FIGS. 8A-B provide results of a hamster challenge study as described in Example 4 using a Toxinotype III PCR-ribotype 027 strain IPP40348 as the challenge strain. Shown in panel A is feces/scoring (diarrheal disease) over time in the vaccine group (with a vaccine that included 160 μg AIOOH) as compared to the placebo control group. Shown in panel B is percent survival overtime in the 160 μg AIOOH vaccine group as compared to the placebo control group.

FIGS. 9A-B provide results of a hamster challenge study as described in Example 4 using a Toxinotype III PCR-ribotype 027 strain CDC13695#7 as the challenge strain. Shown in panel A is feces/scoring (diarrheal disease) over time in the vaccine group as compared to the placebo control group. Shown in panel B is percent survival overtime in the vaccine group as compared to the placebo control group.

FIG. 9C-D provide results of a hamster challenge study as described in Example 4 using a Toxinotype III PCR-ribotype SP041 strain as the challenge strain. Shown in panel A is feces/scoring (diarrheal disease) over time in the vaccine group as compared to the placebo control group. Shown in panel B is percent survival overtime in the vaccine group as compared to the placebo control group.

FIGS. 10A-B provides results of a hamster challenge study as described in Example 4 using a Toxinotype IV PCR-ribotype 023 strain as the challenge strain. Shown in panel A is feces/scoring (diarrheal disease) over time in the vaccine group as compared to the placebo control group. Shown in panel B is percent survival overtime in the vaccine group as compared to the placebo control group.

FIGS. 11A-B provides results of a hamster challenge study as described in Example 4 using a Toxinotype V PCR-ribotype 078 strain as the challenge strain. Shown in panel A is feces/scoring (diarrheal disease) over time in the vaccine group as compared to the placebo control group. Shown in panel B is percent survival overtime in the vaccine group as compared to the placebo control group.

FIG. 12A-C provide results of a hamster challenge study as described in Example 4 using a Toxinotype VIII PCR-ribotype 017 strain as the challenge strain. Shown in panel A is feces/scoring (diarrheal disease) over time in the vaccine group as compared to the placebo control group. Shown in panel B is percent survival overtime in the vaccine group as compared to the placebo control group. Shown in panel C is percent body weight change overtime in the vaccine group as compared to the placebo control group.

### SUMMARY OF THE DISCLOSURE

This disclosure provides compositions including C. difficile antigens for use as therapeutic and/or protective immunogenic compositions (e.g., vaccines) against multiple C. difficile strains/toxinotypes, especially against both those that express C. difficile binary toxin (“CDT”; e.g., from its subunits CDTa and/or CDTb) and those that do not. In preferred embodiments, the composition does not include CDT or a subunit thereof (or any immunogen thereof). Other embodiments are provided in this disclosure, as will be apparent to one of ordinary skill in the art.

### DETAILED DESCRIPTION

This disclosure provides methods for immunizing a host against multiple C. difficile strains and/or “toxinotypes” (i.e., expressing Toxin A (e.g., expressed from the TcdA gene), Toxin B (e.g., expressed from the TcdB gene), and/or expressing C. difficile binary toxin (“CDT”/“binary toxin”, expressed from the CDTa and/or CDTb genes), or not expressing any one or more of those proteins). A strain and/or toxinotype expressing Toxin A is identified herein as “A⁺”; a strain not expressing Toxin A is identified herein as “A⁻”. A strain and/or toxinotype expressing Toxin B is identified herein as “B⁺”; a strain not expressing Toxin B is identified herein as “B⁻”. A strain and/or toxinotype expressing CDT is identified herein as “CDT⁺”; a strain not expressing CDT is identified herein as “CDT⁻”. A strain and/or toxinotype may be characterized by its expression of any one or more of these markers in any combination (e.g., A⁺B⁺CDT⁺, A⁺B⁺CDT⁻, A⁺B⁻CDT⁺, A⁺B⁻CDT⁻, A⁻B⁺CDT⁻, A⁻B⁻CDT⁺ and the like). For example, Toxinotype O is A⁺B⁺CDT⁻, Toxinotype III is A⁺B⁺CDT⁺, Toxinotype IV is A⁺B⁺CDT⁺, Toxinotype V is A⁺B⁺CDT⁺, and Toxinotype VIII is A⁻B⁺CDT⁻.

The various types of C. difficile toxinotype may be identified using, for instance, methods for toxinotype determination such as those based on restriction fragment length polymorphism (RFLP) of the PaLoc region (toxin expression locus) using the polymerase chain reaction (PCR) to identify the A3 fragment of toxin gene tcdA and the BI fragment of toxin gene tcdB. Some strains may exhibit minor changes in the TcdA and/or TcdB genes as compared to reference strain VP110463 (vaccine strain ATCC43255), for instance. PCR ribotypes may also be determined by exploiting the variability of the intergenic spacer region (ISR) between the 16S and 23S ribosomal DNA (rDNA). The variability, in combination with multiple copies of rDNA present in the genome, results in the amplification of various amplicons after PCR amplification in different strains. To date, PCR ribotyping is capable of identifying more than 400 distinct PCR ribotypes. Multi-Locus-Sequence-based typing methods may also be used in which DNA fragments approximately ranging between 300 and 500 bp and representing seven housekeeping genes (MLST 7HG) are sequenced. This method allows for the identification of clonal complexes; at least five lineages have been identified by this method. The compositions described herein may be used to vaccinate, therapeutically or protectively, against any one or more strains and/or toxinotypes identified by any one or more of these techniques, or any others that may be available to those of ordinary skill in the art.

Also provided are methods for preparing clostridial toxoids, clostridial toxoids prepared by these methods and compositions comprising these toxoids. Of particular interest herein are C. difficile Toxins A and/or B and/or derivatives thereof (e.g. genetically detoxified versions, truncated forms, and the like). For the purposes of this disclosure, Toxin A and/or Toxin B may include any C. difficile toxin that may be identified as Toxin A and/or Toxin B using standard techniques in the art. Exemplary techniques may include, for instance, immunoassays such as ELISA, dot blot or in vivo assays. Reagents useful in making such identifications may include, for instance, anti-Toxin A rabbit polyclonal antisera (e.g., Abcam®Product No. ab35021 or Abcam® Product No. ab93318) or an anti-Toxin A mouse monoclonal antibody (e.g., any of Abcam® Product Nos. ab19953 (mAb PCG4) or ab82285 (mAb B618M)), anti-Toxin B rabbit polyclonal antisera (e.g., Abcam® Product No. ab83066) or an anti-Toxin B mouse monoclonal antibody (e.g., any of Abcam® Product Nos. ab77583 (mAb B428M), ab130855 (mAb B423M), or ab130858 (mAb B424M)) (all available from Abcam® (Cambridge, Mass.)). Provided herein are methods for producing a C. difficile toxoid composition that is stable at high temperature (e.g., 37° C.) and contains low amounts of formaldehyde by one or more of the steps of: 1) providing a C. difficile culture comprising Toxin A and Toxin B; 2) purifying Toxin A and Toxin B from the culture to provide separate compositions of each toxoid; 3) inactivating the purified Toxin A and the purified Toxin B by incubation with about any of 0.15% to about 0.5% formaldehyde (w/v) (e.g., about any of 0.2% to 0.8%, such as about 0.2% (e.g., about 0.21%) for Toxoid A and/or about 0.4% (e.g., about 0.42%) for Toxoid B) at an appropriate temperature (e.g., about any of 17 to 32° C. (e.g., about 25° C.)) for an appropriate amount of time (e.g., about two to about 21 days) (e.g., such that the respective toxin is inactivated into the corresponding toxoid) to generate Toxoid A and Toxoid B compositions, respectively; and, 4) combining the toxoids to produce a toxoid immunological composition and/or vaccine that contains only a “residual amount” of formaldehyde (e.g., about any of 0.0001% to 0.025% such as about any of 0.001%, 0.002%, 0.003%, 0.004%, 0.005%, 0.006%, 0.007%, 0.008%, 0.01%, 0.016%, 0.02% or 0.025% (w/v) (preferably about either of 0.004% or 0.008%)). While the amount of formaldehyde contained in the compositions is typically referred to in terms of a percentage of the composition (weight/volume (“w/v”)), it may be important to adjust the stoichiometry based on certain factors such as protein concentration. For instance, a suitable concentration of formaldehyde as contemplated herein is one that will provide intermolecular crosslinks within individual Toxin A and/or Toxin B polypeptides without also substantially crosslinking the polypeptides to one another (e.g., without producing intermolecular crosslinks). As shown in the Examples, a composition comprising 0.5 mg/ml Toxin A may only require 0.21% (w/v) formaldehyde. However, a composition comprising a higher concentration of Toxin A may require a higher or lower concentration of formaldehyde to produce the required intramolecular crosslinks (e.g., toxoiding) without also producing a substantial amount of intermolecular crosslinks. The same principle may apply to the toxoiding of Toxin B. Suitable conditions for a particular composition may be determined by one of ordinary skill in the art using the techniques described herein or as may be available in the art. For instance, whether a particular amount of formaldehyde is effective for toxoiding a particular toxin in a composition may be determined using any one or more of the cytotoxicity assays, anion exchange chromatography, size exclusion chromatography, amine content analysis, antigenicity and immunogenicity assays described in the Examples section.

It should also be understood that while formaldehyde is used herein, other similar agents may be substituted therefor as may be determined by one of ordinary skill in the art. For instance, in some embodiments, formaldehyde may be substituted by glutaraldehyde. While different concentrations may be required to make such a substitution, suitable conditions for such a substitution may be determined using the techniques described herein (e.g., any one or more of the cytotoxicity assays, anion exchange chromatography, size exclusion chromatography, amine content analysis, antigenicity and immunogenicity assays described in the Examples section).

In certain embodiments, Toxin A may be mixed for an appropriate amount of time (e.g., about any of one to 60 minutes, such as ten minutes) with an appropriate amount of formaldehyde (e.g., about 0.2%) formaldehyde to produce Toxoid A and then incubated at an appropriate temperature (e.g., about 25° C.) for an appropriate amount of time (e.g., about two to 21 days, such as any of about six to 12 days (e.g., about six days)). In some preferred embodiments, as shown in the Examples herein, Toxin A may be converted to Toxoid A by incubating Toxin A in a formulation comprising about 0.21% (w/v) formaldehyde at about 25° C. for about six to about 12 days. In certain embodiments, Toxin B may be mixed for an appropriate amount of time (e.g., about any of one to 60 minutes, such as ten minutes) with an appropriate amount of formaldehyde (e.g., about 0.42%) and then incubated at an appropriate temperature (e.g., about 25° C.) for an appropriate amount of time (e.g., about two to 30 days, such as any of about 13-21 days (e.g., about 21 days)) to produce Toxoid B. In some preferred embodiments, as shown in the Examples herein, Toxin B may be converted to Toxoid B by incubating mixing Toxin B in a formulation comprising about 0.42% (w/v) formaldehyde at about 25° C. for about 13 to about 20 days. The formaldehyde may be introduced (e.g., aseptically) to a desired amount into a solution comprising Toxin A or Toxin B from a stock solution of 37% formaldehyde, followed by incubation for a period of time (e.g., five to ten minutes) and storage for an appropriate temperature and time (e.g., 2-8° C. for multiple days). In certain embodiments, purified Toxin A and purified Toxin B may be combined and then mixed for an appropriate amount of time (e.g., about any of one to 60 minutes, such as ten minutes) with an appropriate amount of formaldehyde (e.g., about 0.42%) and then incubated at an appropriate temperature (e.g., about 25° C.) for an appropriate amount of time (e.g., about two to 30 days, such as any of about 13-21 days (e.g., about 21 days) to produce Toxoids A and B. The toxoids may be contained in a suitable buffer (e.g., about any of 20-150 mM phosphate (e.g., 100 mM), pH 7.0). The Toxoid A and Toxoid B compositions may then be combined in a suitable buffer (e.g., by diafiltration into an appropriate buffer such as 20 mM citrate, pH 7.5, 5%-8% sucrose (e.g., 8%)) to produce a Toxoid A/B immunological composition and/or vaccine (e.g., which may be collectively referred to herein as “composition”). Such compositions may also be prepared in lypohlized form using standard techniques. Thus, in some embodiments, the toxoid immunological composition may be in lyophilized form which may contain, for example, a higher concentration of formaldehyde than a composition reconstituted therefrom (e.g., the drug product). For instance, the lyophilized composition may comprise about 0.016% formaldehyde (w/v) but after reconstitution for administration to a host, the composition (e.g., drug product) may comprise less than 0.016% formaldehyde (w/v) (e.g., about any of 0.001%, 0.002%, 0.003%, 0.004%, 0.005%, 0.006%, 0.007%, 0.008%, 0.01 (w/v)). In some embodiments, then, the Toxoid A/B immunological composition and/or vaccine (e.g., “drug product”) may comprise about any of 0.0001% to 0.025% formaldehyde (w/v) (e.g., about any of 0.001%, 0.002%, 0.004%, 0.005%, 0.006%, 0.007% 0.008%, 0.01%, 0.016%, 0.02% or 0.025% (w/v)) (e.g., “residual formaldehyde”). The inclusion of residual formaldehyde in the drug product has been found to be especially beneficial in that it may reduce and/or prevent reversion of Toxoid A and/or Toxoid B to Toxin A or Toxin B, respectively, where the composition is maintained at higher temperature (e.g., above 4° C. such as room temperature or 37° C., for instance) for a period of time (e.g., about six weeks). It is noted that, in some instances, the amount of formaldehyde may be increased to reduce toxin inactivation time. The final composition (e.g, the immunological composition, vaccine) will include only a residual amount of formaldehyde. As shown in the Examples, these processes surprisingly provide immunological Toxoid A/B-containing compositions having favorable biochemical and functional properties.

In certain embodiments, it may be beneficial to, at any point in the methods described herein, regulate the amount of certain buffer components that may interfere with the functionality of formaldehyde therein. For instance, TRIS has an amine group that can effectively compete with the protein for formaldehyde mediated modification, thereby lowering the effective formaldehyde concentration in the reaction mixture. It may therefore be beneficial to maintain the amounts of TRIS in compositions in which toxins and/or toxoids are produced at a low level. For instance, the residual TRIS values in the toxin preparations may be lowered to more suitable levels (e.g., below about 1 to about 5 μg/ml (e.g., 1 μg/ml (e.g., below limit of detection) or 5 μg/ml). As shown in the Examples, the residual TRIS values in the toxin preparations may surprisingly be lowered to more suitable levels (e.g., below 1 μg/ml) by diafiltering purified toxin A and/or purified toxin B into 25 mM Tris (e.g., to remove MgCl₂) and then into a phosphate buffer (e.g., 100 mM PO₄, pH 7) using, for instance, tangential flow filtration (e.g., with flat stock Millipore PES50K) (e.g., as opposed to hollow-fiber or other type of membrane). The resulting lower concentration of TRIS may, in some embodiments, allow one to more effectively adjust the amount of formaldehyde required to effect the toxoiding process. Other embodiments may involve, for instance, using buffers that do not contain amine groups (e.g., MEM, acetate, citrate) and/or a pH-controlled aqueous solution (e.g., saline or water to which acid or base may be added).

Thus, in some preferred embodiments, Tris may be replaced by another buffer such as a phosphate buffer. For instance, as described in the Examples, clarified C. difficile culture filtrate may be processed (e.g., concentrated and diafiltered such as by tangential flow filtration) into a Tris buffer (e.g., 50 mM Tris/NaCl/0.2 mM EDTA/1 mM DTT, pH 7.5). The resulting solution may then be filtered (e.g., using a membrane filter), ammonium sulfate concentration adjusted to about an appropriate amount (e.g., to about 0.4M) and then a further filtration may be performed (e.g., using a membrane filter). This aqueous solution, containing C. difficile toxin A and toxin B, may then be subjected to hydrophobic interaction chromatography and the toxins bound to a size exclusion (e.g., sepharose) column that may be washed with a Tris buffer. The C. difficile toxins may then be eluted with a Tris buffer containing DTT and IPA, pooled and adjusted to a conductivity of about 9 mS or less using WFI. These C. difficile toxins (in pooled elutate) may then be further purified by another method such as anion exchange chromatography involving the equilibration with a Tris buffer. Toxin A may then be eluted with a low-salt Tris buffer and toxin B with a high salt Tris buffer. The solutions containing purified toxin A or purified toxin B may each then be concentrated and diafiltered into a phosphate buffer such as 100 mM PO₄, pH 7 (where the residual TRIS values are preferably below about 1 to about 5 μg/ml). It has been found that lower concentrations of phosphate (e.g., 20 mM) may not be appropriate and may lead to increased multimerization (which should be minimized where possible). Thus, preferred suitable phophate buffers may include any concentration of phosphate from above about 20 mM up to about 200 mM such as, for instance, about any of 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 mM. As shown in the Examples herein, then, Toxin A may be converted to Toxoid A by mixing Toxin A with a formulation comprising about 0.21% (w/v) formaldehyde in 100 mM PO₄, pH 7 at about 25° C. for about six days. And in some preferred embodiments, as shown in the Examples herein, Toxin B may be converted to Toxoid B by mixing Toxin B with a formulation of about 0.41% (w/v) formaldehyde in 100 mM PO₄, pH 7 at about 25° C. for about 13 days. Other suitable buffers are also contemplated as would be understood by those of ordinary skill in the art.

One of ordinary skill in the art may determine whether a particular condition (e.g., buffer (or component thereof), time, temperature) is suitable for use in preparing and/or maintaining Toxoid A and/or Toxoid B compositions by assaying the same to determine whether the characteristics of the compositions are acceptable. For instance, the compositions may be tested using a cytooxicity assay (e.g., using the IMR-90 cell line (see, e.g., the Examples) or Veto cells), anion exchange high-performance liquid chromatography (AEX-HPLC), size exclusion high-performance liquid chromatography (SEC-HPLC), enzyme-linked immunosorbent assay (ELISA), concentration measured using absorbance at 280 nm, reversion analysis (see, e.g., the Examples), and/or in vivo potency assay (e.g., hamster potency assay as described in the Examples). Compositions prepared under favorable conditions may typically exhibit any one or more of: little to no cytotoxicity for the cells monitored in cytotoxicity assays; AEX-HPLC and/or SEC-HPLC chromatograms showing little to no (or at least less under one condition versus another, less being preferable) multimerization of toxoid(s); an ELISA/A280 value closer to 1 (e.g., as compared to compositions prepared under unfavorable conditions that may typically exhibit ELISA/A280 values further from 1); little to no reversion from toxoid to toxin during the testing period; and/or immunogenicity during in vive assays (e.g., a. Log 10 titer of 4.8 or higher in a hamster potency assay). Other methods may also be used to make these determinations as may be determined by those of ordinary skill in the art.

The methods described herein are applicable to toxins from virtually any strain of C. difficile. Preferred strains of C. difficile are strains which produce Toxin A and/or B and include for example, but are not limited to strains of toxinotype 0 (A⁺B⁺CDT⁻; e.g., VPI0463/ATCC43255 (PCR ribotype 087), 630; PCR ribotypes 001, 002, 012, 014/020, among others), III (A⁺B⁺CDT⁺; e.g, 027/NAP/1, NAP1/027/BI17, IPP4038 (PCR ribotype 027), CDC 13695#7 (PCR ribotype 027), SP041 (PCR ribotype 027)), IV (A⁺B⁺CDT⁺; e.g., NK91 (PCR ribotype 023)), V (A⁺B⁺CDT⁺; e.g., BAA 1875 (PCR ribotype 078126)) and I or VIIII (A⁻B⁺CDT⁻; e.g., ATCC43598 (PCR ribotype 017)). In some embodiments, the methods provide for immunization (e.g., therapeutically or protectively) against all of the toxinotypes 0, III, IV, V and VIII. In some embodiments, the methods provide for immunization against both CDT⁺ and CDT⁻ strains/toxinotypes. Methods are also applicable to C. difficile toxins produced using recombinant methods, except that the art has recognized that toxoids prepared from recombinant toxins may not provide immunity against CDT⁺ C. difficile strains unless the CDT subunits are included in a vaccine (see, e.g., WO 2013/112867). The toxins (e.g., Toxin A and/or Toxin B) may be purified from culture filtrates of C. difficile using methods known in the art (e.g., U.S. Pat. No. 6,669,520). Exemplary methods of purifying toxins from culture filtrates of C. difficile are described in the Examples herein. Preferably the toxins have a purity of about any of 75%, 80%, 85%, 90%, 95%, 99% or more. The toxins may be inactivated together or separately. For example, the purified toxins may be mixed at a desired Toxin A: Toxin B ratio (e.g., 3:1, 3:2, 5:1, 1:5) and then inactivated or may be inactivated individually. Preferably the toxins are individually inactivated to produce toxoids. The term “toxoid” is used herein to describe a toxin that has been partially or completely inactivated by chemical treatment. A toxin is said to be inactivated if it has less toxicity (e.g., 100%, 99%, 95%, 90%, 80%, 75%, 60%, 55%, 50%, 25% or 10% or less toxicity) than untreated toxin, as measured, for example, by an in vitro cytotoxicity assay or by an in vivo assay. As disclosed herein, the toxins are inactivated using formaldehyde treatment. Other possible chemical means include for example, glutaraldehyde, peroxide, ß-priopiolactone or oxygen treatment.

Inactivation may be carried out by incubating the toxin(s) with an amount of formaldehyde that prevents reversion of a toxoid into a toxin. Reversion may be prevented by including in a buffer comprising purified Toxin A or Toxin B a suitable amount of formaldehyde. The amount of formaldehyde in the buffer may be adjusted to maintain an appropriate concentration of formaldehyde to prevent reversion. To this end, a residual concentration of formaldehyde may be included in the buffer (and/or pharmaceutical composition). A residual concentration of formaldehyde is one that prevents reversion and/or presents a low risk of side effects to one to whom a composition described herein is administered. For instance, a residual formaldehyde concentration may range from about any of 0.0001% to 0.025% formaldehyde (w/v) (e.g., about any of 0.004%, 0.008%, 0.016%, or about 0.01%), about 0.001% to about 0.020% (w/v), about 0.004% to about 0.020% (w/v) (e.g., about 0.016%±0.04%), or about 0.004% to 0.010% (w/v) (e.g., about 0.008%), among other ranges. Prevention of reversion is typically found where no detectable cytotoxicity is observed following storage at 37° C. by the in vitro assay as described herein (see, e.g., the cytotoxicity assays in the Examples). “Substantial” prevention of reversion typically means that 10% or less of the toxoid reverts into toxin following storage at 37° C. by the in vitro assay described in the Examples. A suitable in vitro cytotoxicity assay may be the cell-based florescence assay using, for instance, Vero cells. Another suitable in vitro cytotoxicity assay may be performed using IMR90 cells (e.g., ATCC® Accession No. CCL-186). Toxicity of the test material (e.g., toxoid) may be determined as the minimum concentration at which 50% of the cells become rounded as compared to their normal striated morphology (e.g., the MC-50). As described in the Examples herein, vaccine compositions comprising toxoids made by the methods described herein and formaldehyde of 0.008% or less showed no detectable cytotoxicity following storage at 37° C. by in vitro assay. Physicochemical analysis (e.g., anion exchange chromatography) may also be used to ascertain reversion but the in vitro cytotoxicity assay may be more informative. The potency of the toxoids may also be measured by a hamster in vivo potency assay which measures the mean of log 10 anti-Toxin A or anti-Toxin B IgG titer.

In some embodiments, the appropriate amount of formaldehyde may be added to the toxins from a solution of 37% formaldehyde. The toxins are preferably in a suitable buffer solution (e.g., 100 mM sodium phosphate buffer, pH 7.0) prior to the addition of formaldehyde. Toxin concentration therein may be, for example, about 0.1 to about 5 mg/mL (e.g., 0.5 mg/mL). To begin the inactivation process, the toxins may initially be mixed with suitable concentration of formaldehyde (e.g., from about 0.1% to about 0.6%) for a suitable period of time (e.g., ten minutes). For example, purified Toxin A (0.5 mg/ml purified Toxin A in 100 mM sodium phosphate, pH 7.0) may be mixed in about 0.2% formaldehyde for about ten minutes. And purified Toxin B (e.g., 0.5 mg/ml purified Toxin B in 100 mM sodium phosphate, pH 7.0) may be mixed in about 0.4% formaldehyde for about ten minutes. Such mixtures may then be filtered (e.g., using 0.2 μm membrane filer) to remove small protein aggregates that may affect the protein concentration by absorbance at 280 nm (e.g., allowing for precise formulation of the pharmaceutical composition at the intended Toxoid A:Toxoid B ratio). Inactivation may then be continued by incubating the mixture for about one to about 21 days (e.g., about two days, about six days, or about 13 days). For instance, the Toxin A mixture may be incubated in 13 days or less (e.g., about two days or about six days) at a suitable temperature (e.g., about 25° C.). And the Toxin B mixture may be incubated for 21 days or less (e.g., about two days, about six days, or about 13 days) at a suitable temperature (e.g., about 25° C.). In this way, preparations of Toxoid A and Toxoid B may be provided. Such preparations typically comprise at least about any of 90%, 95%, 99% or 100% toxoid (e.g., inactivated toxin).

Although these toxoid preparations may be mixed directly with buffer, preferably the preparations are concentrated and diafiltered into an appropriate buffer solution. Preferably, concentration and diafiltration is done using tangential flow filtration to minimize protein shear while ensuring removal of formaldehyde and exchange into buffer. The buffer preferably includes at least one or more pharmaceutically acceptable excipients that increase the stability of the toxoids and/or delay or decrease aggregation of the toxoids. Excipients suitable for use include for example but are not limited to sugars (e.g., sucrose, trehalose) or sugar alcohols (e.g., sorbitol), and salts (sodium chloride, potassium chloride, magnesium chloride, magnesium acetate) or combinations thereof. Additionally, suitable excipients may be any of those described in, for example, US Pat. Pub. 2011/045025 (Ser. No. 12/667,864). Following inactivation, the solutions of inactivated toxins (i.e., toxoids) may be concentrated and/or ultrafiltered and/or diafiltered and stored in an appropriate buffer (such as, for example, but not limited to, about 5 to about 100 mM (e.g., about any of 5, 10, 15, 20, 25, 30, 35, 40, 45, 50, 55, 60, 65, 70, 75, 80, 85, 90, 95, or 100 mM citrate, phosphate, glycine, carbonate, bicarbonate, or the like, buffer) at a pH 8.0 or less (e.g., 6.5-7.7 such as about any of 6.5, 6.6, 6.7, 6.8, 6.9, 7.0, 7.1, 7.2, 7.3, 7.4, 7.5, 7.6, 7.7, 7.8, 7.9 or 8.0) (e.g., 20 mM citrate, pH 7.5) that prevents, or substantially prevents, reversion of the toxoids into a cytotoxic form (e.g., into a toxin). An exemplary buffer may be, for instance, 20 mM citrate, pH 7.5, 5%-8% sucrose, containing a suitable amount of formaldehyde (e.g., 0.016% (w/v)). Other buffers and the like may also be suitable, as would be understood by those of ordinary skill in the art.

The toxoids may be formulated for use as pharmaceutical compositions (e.g., immunogenic and/or vaccine compositions). For example, compositions comprising the C. difficile toxoids can be prepared for administration by suspension of the toxoids in a pharmaceutically acceptable diluent (e.g., physiological saline) or by association of the toxoids with a pharmaceutically acceptable carrier. Such pharmaceutical formulations may include one or more excipients (e.g., diluents, thickeners, buffers, preservatives, adjuvants, detergents and/or immunostimulants) which are known in the art. Suitable exicipents will be compatible with the toxoid and with the adjuvant (in adjuvanted compositions), with examples thereof being known and available to those of ordinary skill in the art. Compositions may be in liquid form, or lyophilized (as per standard methods) or foam dried (as described, e.g., in U.S. Pat. Pub. 2009/110699). An exemplary lyophilized vaccine composition may comprise for example, Toxoids A and B, 20 mM citrate, 8% sucrose, 0.016% formaldehyde, pH 7.5.

To prepare a vaccine for administration, a dried composition may be reconstituted with an aqueous solution such as, for example, water for injection, or a suitable diluent or buffer solution. In certain examples, the diluent includes formaldehyde as described herein. The diluent may include adjuvant (e.g., aluminum hydroxide) with or without formaldehyde. An exemplary diluent may be an aqueous solution of NaCl and aluminum hydroxide. Such a diluent may be used to reconstitute the dried composition. The pharmaceutical compositions may comprise a dose of the toxoids of about 10 to 150 μg/mL (e.g., any of about 10, 20, 30, 40, 50, 60, 70, 80, 90, 100, 110, 120, 130, 140 or 150 μg/mL). Typically, a volume of a dose for injection is about 0.5 mL or 1.0 mL. Dosages can be increased or decreased as to modulate immune response to be induced in a subject. The toxoids can be administered in the presence or absence of an adjuvant, in amounts that can be determined by one skilled in the art. Adjuvants of use include aluminum compounds, such as aluminum hydroxide, aluminum phosphate and aluminum hydroxyl phosphate. For instance, in animal studies described in the Examples, a composition comprising 5 μg of Toxoid A and Toxoid B (bivalent toxoid) and 20 μg or 160 μg adjuvant (per immunization dose, corresponding to 1/20 of a human dose). Other combinations of toxoids and adjuvants may be suitable as would be understood by those of ordinary skill in the art.

The immunological and/or vaccine compositions can be administered by the percutaneous (e.g., intramuscular, intravenous, intraperitoneal or subcutaneous), transdermal, mucosal route in amounts and in regimens determined to be appropriate by those skilled in the art to subjects that have, or are at risk of developing, symptomatic C. difficile infection. The vaccine can be administered 1, 2, 3, 4 or more times. When multiple doses are administered, the doses can be separated from one another by, for example, one week, one month or several months. Thus, this disclosure also provides methods of eliciting an immune response against the toxins, toxoids, and/or infectious organism comprising the same by administering the pharmaceutical compositions to a host. This may be achieved by administration of the pharmaceutical compositions (e.g., immunogenic compositions and/or vaccines) described herein to the subject to effect exposure of the toxoids to the immune system of the subject. Thus, the immunogenic compositions and/or vaccines may be used to prevent and/or treat symptomatic C. difficile infections.

Compositions may be included in a kit (e.g., a vaccine kit). For example, the kit may comprise a first container containing a composition described herein in dried form and a second container containing an aqueous solution for reconstituting the composition. The kit may optionally include the device for administration of the reconstituted liquid form of the composition (e.g., hypodermic syringe, microneedle array) and/or instructions for use. Such kits are possible since it has been found that compositions as described herein can have good stability and remain non-cytotoxic following storage periods at moderate temperatures (e.g., at about 2-8° C.) and higher temperatures (e.g., at about 15° C., 25° C., 37° C. or higher). In certain examples, as described further below, compositions remained non-cytotoxic (e.g., without evidence of reversion) following storage at 37° C.

Thus, this disclosure provides methods for producing C. difficile toxoids by, for instance, inactivating purified C. difficile Toxin A and/or purified C. difficile Toxin B by incubation with about 0.15%-0.5% formaldehyde (w/v) at about 17-32° C. for about two to about 21 days. In some embodiments, Toxin A may be incubated with about 0.2% formaldehyde at about 25° C. for about two days to produce Toxoid A and/or Toxin B is incubated with about 0.4% formaldehyde at about 25° C. for about 13 days to produce Toxoid B. Compositions comprising Toxoid A and/or Toxoid B prepared by such methods are also provided. Methods are also provided for preparing immunogenic compositions comprising purified C. difficile Toxoid A and purified C. difficile Toxoid B by combining purified C. difficile Toxoid A and purified C. difficile Toxoid B with a composition comprising a residual amount of formaldehyde (e.g., about 0.004%, 0.008%, or 0.016% (w/v)). In some embodiments, the methods may provide compositions of C. difficile Toxoid A and/or purified C. difficile Toxoid B that are stable at 37° C. for up to about six weeks. Thus, in some embodiments, the methods described herein may also comprise inactivating purified C. difficile Toxin A or purified C. difficile Toxin B by incubation with about 0.15%-0.5% formaldehyde (w/v) at about 17-32° C. for about two to about 21 days; and, combining C. difficile Toxoid A and purified C. difficile Toxoid B with a composition comprising a residual amount of formaldehyde. The C. difficile Toxoids A and B compositions prepared by such methods may be stable at 37° C. for up to about six weeks. The residual amount of formaldehyde in such compositions may be about any of 0.004%, 0.008%, or 0.016% (w/v). The composition may also comprise about 20 mM citrate, pH 7.5, 4% to 8% sucrose, and 0.016% formaldehyde. In some embodiments, the composition may be lyophilized. These methods may also comprise providing a C. difficile culture comprising Toxin A and Toxin B and purifying the Toxin A and Toxin B from the culture. C. difficile Toxoids A or B produced in accordance with these method are also provided. In some embodiments, such compositions are vaccines (e.g., provide a protective, prophylactic, and/or therapeutic response against symptomatic C. difficile infection). The compositions (e.g., vaccine compositions) may comprise Toxoid A and Toxoid B in an A:B ratio of 5:1 to 1:5 such as 3:1 or 3:2. In some embodiments, the composition may be lyophilized, freeze dried, spray dried, or foam dried, or in liquid form. Such compositions may comprise one or more pharmaceutically acceptable excipients, a buffer such as a citrate, phosphate, glycine, carbonate, or bicarbonate buffer, or a pH-controlled aqueous solution, and/or one or more sugars (e.g., sucrose, trehalose) and/or sugar alcohol (sorbitol). Other embodiments will be apparent to those of ordinary skill in the art.

In some embodiments, this disclosure provides methods for immunizing a host against C. difficile strains expressing C. difficile binary toxin (CDT) and C. difficile strains not expressing CDT, the method comprising administering to the host an immunogenic composition comprising purified C. difficile Toxin A and purified C. difficile Toxin B inactivated by incubation with formaldehyde (w/v) at about 17-32° C. for about two to about 21 days, wherein Toxin A is inactivated with 0.15%-0.5% formaldehyde (w/v) and Toxin B is inactivated with 0.15%-0.8% formaldehyde (w/v). The strain may be of any Toxinotype and Ribotype. The strain may be of Toxinotype 0, III, IV, V and/or VIII, preferably all of Toxinotypes 0, III, IV, V and VIII. In preferred embodiments, the purified Toxin A and purified C. difficile Toxin B are derived from a C. difficile strain that does not express C. difficile binary toxin (CDT), such as C. difficile strain VPI10463/ATCC43255.

This disclosure also provides methods for inducing antibodies in a host, the antibodies having specificity for one or more C. difficile strains expressing C. difficile binary toxin (CDT) by administering to the host a composition comprising inactivated purified C. difficile Toxin A and inactivated purified C. difficile Toxin B derived from a C. difficile strain that does not express CDT (e.g., C. difficile strain VP110463/ATCC43255). In some embodiments, the antibodies produced following administration of the composition may be neutralizing antibodies as determined by a toxin neutralizing assay (see, e.g., the Examples). In some embodiments, the antibodies may exhibit a relative efficacy (RE) of at least 5.4 as determined using such an assay. In some embodiments, the methods may induce the production of antibodies that neutralize toxin A and/or toxin B produced by a C. difficile strain having a toxinotype selected from the group consisting of 0, I, III, IV, V, VI, VII, VIII, IX, and XII. In some embodiments, the toxinotype 0 strain may have the PCR-ribotype selected from the group consisting of 001, 002, 012, 014, 020, 014/020, 014/020/077, 106, 018, and 053; the toxinotype III strain may have the PCR-ribotype 027 or 075; the toxinotype IV strain may have the PCR-ribotype 023; the toxinotype V strain may have the PCR-ribotype selected from the group consisting of 078, 079, 122, 126, and 078/126; the toxinotype VI strain may have the PCR-ribotype 127 or 66-2; the toxinotype VII strain has the PCR-ribotype 66-2; the toxinotype VIII strain may have the PCR-ribotype 017; the toxinotype IX strain has the PCR-ribotype 019; the toxinotype XII strain may have the PCR-ribotype 056; and/or the C. difficile strain may have PCR-ribotype 046 or 369. In some embodiments, the antibodies may neutralize toxin A and/or toxin B produced by a C. difficile strain having a toxinotype selected from the group consisting of 0, III, IV, V, and VIII, such as strains of each of these toxinotypes (i.e., the antibodies neutralize toxin A and/or toxin B produced by C. difficile strains toxinotype 0, III, IV, V, and VIII). In some such embodiments, the antibodies the toxinotype 0 strain may have the PCR-ribotype 012, the toxinotype III strain may have the PCR-ribotype 027, the toxinotype IV strain may have the PCR-ribotype 023, the toxinotype V strain may have the PCR-ribotype 078, and the toxinotype VIII strain may have the PCR-ribotype 017. In some embodiments, the antibodies may neutralize toxin A and/or toxin B produced a C. difficile strains of toxinotype III, IV and V. In some such embodiments, the toxinotype III strain may have the PCR-ribotype 027, the toxinotype IV strain may have the PCR-ribotype 023, and/or the toxinotype V strain may have the PCR-ribotype 078. Other embodiments are also contemplated by this disclosure as will be understood by those of ordinary skill in the art.

This disclosure also provides methods for immunizing and/or vaccinating a host against one or more C. difficile strains expressing C. difficile binary toxin (CDT) by administering to the host a composition comprising inactivated purified C. difficile Toxin A and inactivated purified C. difficile Toxin B derived from a C. difficile strain that does not express CDT (e.g., C. difficile strain VP110463/ATCC43255). In some such embodiments, the host may be immunized and/or vaccinated, respectively, against one or more C. difficile strains having a toxinotype selected from the group consisting of 0, III, IV, V and/or VIII. In some such embodiments, the host may be immunized and/or vaccinated against one or more C. difficile strains having a toxinotype selected from the group consisting of III, IV and V such as strains of each of such toxinotypes (i.e., the host is immunized and/or vaccinated, respectively against C. difficile strains having the toxinotypes III, IV and V). In some embodiments, the toxinotype III strain may have the PCR-ribotype 027, the toxinotype IV strain may have the PCR-ribotype 023, and the toxinotype V strain may have the PCR-ribotype 078. In some embodiments, significant protection against disease and death caused by C. difficile may be provided to the host results from such methods. In some embodiments, protection (e.g., immunization and/or vaccination) may be determined using the Golden Syrian hamster model. In some embodiments, the composition may be administered to the host at least three times which may be separated by sufficient period of time such as about any of seven days, 10 days, or 14 days (two weeks), and the time between doses may be the same or different. While administration may be by any route of administration although, in some embodiments, the composition may be administered via the intramuscular route. In some embodiments, the survival rate for a group of hamsters following challenge with a CDT⁺ strain of C. difficile is about 58% to about 100%. In some embodiments, protection may be statistically significant. In some embodiments, statistical significance may be determined by the Kaplan-Meier method with log-rank test and/or the bilateral Fisher exact test (e.g., such as, for a group of hamsters: p=0.0001 with the Kaplan Meier log-rank test and p=0.0004 with the bilateral Fisher exact test; p<0.001 with the Kaplan Meier log-rank test and p-value=0.005 with the bilateral Fisher exact test; p-values ≤0.0001 with both the Kaplan Meier log-rank test and the bilateral Fisher exact test; and/or p-value=0.0020 with the Kaplan Meier log-rank test and p=0.0046 with the bilateral Fisher exact test). Other embodiments are also contemplated by this disclosure as will be understood by those of ordinary skill in the art.

Thus, in some embodiments, the composition comprises inactivated purified C. difficile Toxin A and inactivated purified C. difficile Toxin B derived from a C. difficile strain that does not express CDT does not include CDT or a subunit thereof. In some embodiments, the C. difficile Toxin A and Toxin B may be derived from C. difficile Toxinotype 0. In some embodiments, the purified C. difficile Toxin A and purified C. difficile Toxin B may be derived from C. difficile strain VP110463/ATCC43255. In some embodiments, the Toxin A and Toxin B may be inactivated by incubation with formaldehyde at about 15-32° C. for about two to about 21 days and wherein Toxin A may be inactivated with 0.15%-0.5% formaldehyde (w/v) and Toxin B may be inactivated with 0.15%-0.8% formaldehyde (w/v). In some embodiments, the composition may comprise about 0.001% to 0.020% formaldehyde (e.g., about 0.004%, about 0.008%, or about 0.016% formaldehyde). In some embodiments, the Toxoid A and the Toxoid B may be present in the composition in a A:B ratio of 5:1 to 1:5 (e.g., about 3:1 or 3:2). In some embodiments, the composition may be freeze dried, spray dried, or foam dried. In some embodiments, the composition may be in liquid form. In some embodiments, the composition may comprise one or more pharmaceutically acceptable excipients. In some embodiments, the composition may comprise a citrate, phosphate, glycine, carbonate, or bicarbonate buffer, or a pH-controlled aqueous solution and/or one or more sugars and/or sugar alcohols. In some embodiments, the composition may comprise sucrose and/or citrate. In some embodiments, the composition may further comprise an adjuvant such as one comprising aluminum (e.g., aluminum phosphate or aluminum hydroxide). In some embodiments, the composition may comprise from about 20 μg to about 160 μg aluminum hydroxide. Other embodiments are also contemplated by this disclosure as will be understood by those of ordinary skill in the art.

A “purified” toxin typically means that the toxin has been isolated, for example, from culture filtrate and purified at least to some extent using methods known in the art. Exemplary methods of purifying toxins are described herein, for example. In some embodiments, a purified toxin may have a purity of about any of 75%, 80%, 85%, 90%, 95%, 99% or more. Similarly, a “purified” toxoid may be a toxoid that has a purity of about any of 75%, 80%, 85%, 90%, 95%, 99% or more.

The terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto. For instance, the terms “about” or “approximately” may include values +/−10% of the indicated value (e.g., “about 30° C.” may mean any value between 27° C. to 33° C., including but not limited to 30° C.

The terms “subject” and “host” are used interchangeably herein.

The terms “incubating”, “mixing” and “storing” (or synonyms and/or derivatives thereof) may be used interchangeably. For instance, a toxin may be incubated with a solution comprising formaldehyde. Such an incubation may optionally mean, for instance, that the composition is being actively combined by motion (e.g., using a mixing bar of the like) or is being maintained in essentially a stationary state.

Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination).

Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed.

When the terms prevent, preventing, and prevention are used herein in connection with a given treatment for a given disease (e.g., preventing symptomatic infection), it is meant to convey that the treated subject either does not develop a clinically observable level of the disease at all, or develops it more slowly and/or to a lesser degree than he/she would have absent the treatment. These terms are not limited solely to a situation in which the subject experiences no aspect of the condition whatsoever. For example, a treatment will be said to have prevented a symptomatic infection if it results in the subject experiencing fewer and/or milder symptoms of the disease than otherwise expected. A treatment can “prevent” symptomatic infection by resulting in the subject displaying only mild overt symptoms of the infection; it does not imply that there must have been no penetration of any cell by the infecting microorganism.

Similarly, reduce, reducing, and reduction as used herein in connection with the risk of infection with a given treatment (e.g., reducing the risk of a symptomatic C. difficile infection) typically refers to a subject developing an infection more slowly or to a lesser degree as compared to a control or basal level of developing an infection in the absence of a treatment (e.g., administration or vaccination using toxoids disclosed). A reduction in the risk of symptomatic infection may result in the subject displaying only mild overt symptoms of the infection or delayed symptoms of infection; it does not imply that there must have been no penetration of any cell by the infecting microorganism.

All references cited within this disclosure are hereby incorporated by reference in their entirety. Certain embodiments are further described in the following examples. These embodiments are provided as examples only and are not intended to limit the scope of the claims in any way.

### EXAMPLES

The following examples are provided solely for purposes of illustration and are not intended to limit the scope of the disclosure. Changes in form and substitution of equivalents are contemplated as circumstances may suggest or render expedient. Although specific terms have been employed herein, such terms are intended in a descriptive sense and not for purposes of limitations. Methods of molecular genetics, protein biochemistry, and immunology used, but not explicitly described in this disclosure and these Examples, are amply reported in the scientific literatures and are well within the ability of those skilled in the art.

### Example 1

A C. difficile working seed (strain VP110463/ATCC43255) was used to inoculate preconditioned culture medium comprising soy peptone, yeast extract, phosphate buffer and sodium bicarbonate, pH 6.35-7.45 (SYS medium) and scaled up from a 4 mL Working Cell Bank (WCB) vial to a 160 L culture. Upon reaching the desired density and the 10-12 hour incubation period, the entire 160 L of culture was processed for clarification and 0.2 μm filtration. The culture from one more production fermentor was harvested and subjected to membrane filtration (e.g., using a Meisner membrane filter) to remove C. difficile cells and cell debris impurities. The resulting clarified culture filtrate was concentrated and diafiltered by tangential flow filtration into 50 mM Tris/NaCl/0.2 mM EDTA/1 mM DTT, pH 7.5. The resulting solution was filtered using a membrane filter, the concentration of ammonium sulfate was increased (e.g., to about 0.4M) and then a further filtration was performed (e.g., using a membrane filter). This aqueous solution contained C. difficile toxin A and toxin B. The aqueous solution was subjected to hydrophobic interaction chromatography. The C. difficile toxins were bound to a sepharose column. The column was washed with a Tris buffer and two fractions of the C. difficile toxins were eluted with a Tris buffer containing DTT and IPA. The two toxin fractions eluted from HIC were pooled and the conductivity adjusted to 9 mS or less using WFI. The C. difficile toxins (in pooled elutate) were further purified by anion exchange chromatography. The eluted aqueous solution was passed through an anion exchange column to bind toxins to column. The column was equilibrated with a Tris buffer and toxin A eluted with a low-salt Tris buffer and toxin B was eluted with high salt Tris buffer. Purified toxin A and purified toxin B were each concentrated and diafiltered into 100 mM PO₄, pH 7. Protein concentration was about 0.5 mg/mL and purity of each toxin was 90% or greater.

A 37% formaldehyde solution was added aseptically to each of the Toxin A diafiltrate and the Toxin B diafiltrate to obtain a final concentration of 0.42%. The solutions were mixed and then stored at 2-8° C. for 18-22 days. Following inactivation, the toxin diafiltrates were dialyzed into formulation buffer (20 mM citrate/5% sucrose, pH 7.5). The formaldehyde concentration was adjusted as required by adding 37% formaldehyde solution. Toxoids A and B were combined in a ratio of 3:2 (A:B) by weight and lyophilized. The lyophilized product comprised Toxoid A (0.24 mg/mL), Toxoid B (0.16 mg/mL), 20 mM sodium citrate, 5% (w/v) sucrose and the indicated concentration of formaldehyde.

A reversion analysis was performed to observe the potential reversion over a 6 week period at 37° C. Compositions comprising Toxoid A and Toxoid B were formulated with differing amounts of residual formaldehyde (0%, 0.008%, and 0.016% (w/v)), stored at either 37° C. or 4° C., and tested via cytotoxicity assay weekly for 6 weeks. Data from these studies are set out in Table 1. At 4° C., the drug product passes reversion analysis even with no residual formaldehyde added. However, at 37° C., 0.016% residual formaldehyde is needed to pass the reversion test.

TABLE 1
Reversion Analysis of Drug Product Stored at 37° C.

| | Day 7 | Day 14 | Day 21 | Day 28 | Day 35 | Day 42 |
|---|---|---|---|---|---|---|
| 4° C. | | | | | | |
| 0% | − | − | − | − | − | − |
| 0.008% | − | − | − | − | − | − |
| 0.016% | − | − | − | − | − | − |
| 37° C. | | | | | | |
| 0% | + | + | + | + | + | + |
| 0.008% | − | + | + | − | − | − |
| 0.016% | − | − | − | − | − | − |

* − = no cytotoxicity detected; + = cytotoxic

### Example 2

The experiments described herein were performed to identify a toxoiding method that would provide toxoids stable at 37° C. A C. difficile working seed (strain VP110463/ATCC43255) was used to inoculate preconditioned culture medium (comprising soy peptone, yeast extract, phosphate buffer and D-sorbitol, pH 7.1-7.3) in a sterile disposable bag and culture was incubated at 35-39° C. until target OD was achieved. The 30 L Seed 1 culture was used to inoculate culture medium in a 250 L sterile disposable culture bag and culture was incubated at 35-39° C. until target OD is achieved. The Seed 2 culture was used to inoculate 1000 L sterile disposable culture bags and culture was incubated at 35-39° C. until target OD is achieved. The culture from one more production fermentor was harvested and subjected to depth filtration (e.g., using a Pall Depth 700p/80p/0.2 um 0.02 msq/L) to remove C. difficile cells and cell debris impurities and simultaneously cooled (e.g., about 37° C.-19° C.) to limit protease activity. The resulting clarified culture filtrate was concentrated and diafiltered by tangential flow filtration using flat stock Millipore and at a temperature of about 4° C. (for reduced protease activity) into 25 mM Tris/50 mM NaCl/0.2 mM EDTA, pH 7.5-8.0 and with no added DTT. The resulting solution was filtered using a membrane filter, the concentration of ammonium sulfate was increased (e.g., to about 0.9M) and then a further filtration was performed (e.g., using a membrane filter). This aqueous solution contained C. difficile toxin A and toxin B. The aqueous solution was subjected to hydrophobic interaction chromatography. The C. difficile toxins were bound to a butyl Sepharose resin (such as e.g., GE Butyl S FF Sepharose). The column was washed with 0.9 mM ammonium sulphate 25 mM Tris, pH 8.0 and C. difficile toxins were eluted with 25 mM Tris, pH 8.0 and conductivity adjusted to 7 mS or less using WFI. The C. difficile toxins (in elutate) were further purified by anion exchange chromatography. The eluted aqueous solution was passed through an anion exchange column (e.g., Tosoh Q 650 M) to bind toxins to column. The column was equilibrated with 25 mM Tris pH 7.5 and toxin A was eluted with 27 mM MgCl₂ in 25 mM Tris, pH 8.0, and toxin B was eluted with 135 mM MgCl₂ in 25 mM Tris, pH 8.0. Purified toxin A and purified toxin B were each concentrated and first diafiltered into 25 mM Tris (e.g., to remove MgCl₂) and then into 100 mM PO₄, pH 7. Average yield of toxin A was about 0.021 g pure toxin/L fermentation (UV280) and purity as evaluated by SDS Page was about 97.2% on average. Average yield of toxin B was about 0.011 g pure toxin/L fermentation (UV280) and purity as evaluated by SDS Page was about 93.9% on average. The toxins generated from this process exhibit a purity of 90% or higher and also show a decrease in the matrix residuals (e.g., tris(hydroxymethyl)aminomethane (TRIS)) left behind from prior process steps. The residual TRIS values in the toxin matrix from the process substantially as described in Example 1 varied ˜100-800 μg/ml where as residual TRIS values in the toxin matrix from the purification process described in this example are below 1 μg/ml (i.e., below limit of detection). In regards to the toxoiding reaction with formaldehyde, TRIS has an amine group that can effectively compete with the protein for formaldehyde mediated modification, thereby lowering the effective formaldehyde concentration in the reaction mixture. Accordingly, data suggests that toxoiding kinetics for the toxoids made by this process are faster as compared to kinetics for the toxoids prepared by the process described in Example 1.

A study was performed on the toxoiding process with respect to temperature and formaldehyde concentration and analyzed as a function of the toxoiding incubation period. The objective was to develop a robust toxoiding process that provided a better safety profile and better reversion characteristics than the toxoids generated using the earlier process (as described in Example 1) while maintaining the same level of immunogenicity. Toxoiding conditions that would yield a drug product that passes reversion analysis at 37° C. with the least amount of residual formaldehyde was desired. In these experiments, toxin concentrations were fixed at 0.5 mg/ml and all of the reactions were performed in 100 mM sodium phosphate buffer, pH 7.0. The temperatures evaluated for each of toxoiding reactions were 4° C., 15° C. and 25° C. Formaldehyde concentration varied between 0.21% (“0.2%”) or 0.42% (“0.4%”) for toxoid A reactions and varied between 0.42% (“0.4%”) and 0.84% (“0.8%”) for toxoid B reactions. For each of the reaction conditions, toxin concentrations were adjusted to 0.5 mg/ml and were performed at the 100 ml scale. Thirty-seven percent (37%) formaldehyde was then added to reach the targeted concentrations for each of the individual reactions. The reactions were gently stirred for 5-10 minutes and placed in incubators at the targeted temperatures (target temp achieved within 1 hour of incubation). Each of the individual reactions were monitored daily for a period up to 21 days. Samples were pulled and analyzed by cytotoxicity analysis, AEX-HPLC, SEC-HPLC, SDS-PAGE and TNBS assay. At certain time intervals depending on toxoiding conditions, samples were pulled, formulated and animal studies, reversion analysis and ELISA testing was performed.

Kinetic Cytotoxicity Analysis

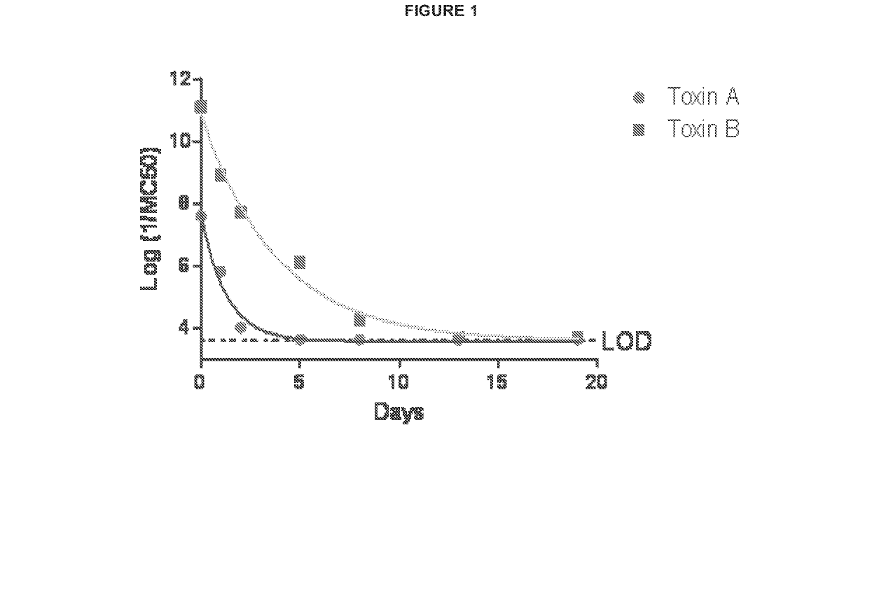

The toxoiding reaction was followed by cytotoxicity analysis and accordingly samples were pulled daily directly from the reaction mixture and submitted for same day analysis. The toxoiding process was followed by cytotoxicity on IMR90 cells and the kinetics of toxoiding was monophasic with Toxin A taking an average of 5±1 days for cytotoxicity neutralization and Toxin B taking close to 13±2 days (falling short of a 3 fold safety margin for the entire reaction). The data obtained using one batch is shown in FIG. 1. The y-axis contains MC50 values which is a reflection of the toxicity of the material and represents the minimum concentration at which the 50% of the cells become rounded in the presence of toxic material instead of their normal striated morphology. The MC 50 values for the two toxins differed by a factor of 1000; B was more cytotoxic with its MC50 value in the low pg/ml range. The absolute MC50 values for the toxoids were unknown as there was no cytotoxicity when tested at the highest concentration of 200 μg/ml in these experiments. The total time period for the inactivation process was 18-21 days.

Data from the cytotoxicity analysis for the toxoiding reactions of Toxin A and Toxin B are shown in Table 2. It portrays the amount of time (in days) needed to show a loss of cytotoxicity for each of the separate reactions of formaldehyde with the toxin. A few general trends are apparent from the data for the toxoiding reactions for Toxins A and Toxins B. As formaldehyde concentration is increased, the time required to inactivate the toxins is decreased. Additionally, as the temperature is increased for the reactions, the time required to inactivate the toxins is also decreased. The data suggests that the rate of toxoiding is accelerated with either an increase in temperature or formaldehyde concentration. Many potential conditions are identified from the kinetic cytotoxicity analysis and data suggests that a 3× safety margin could be achieved by extrapolating the initial loss of cytotoxicity three-fold. For example, Toxin A detoxifies at two days with 0.2% formaldehyde at 25° C., thus, applying an appropriate safety margin would minimally be continuing the reaction for six days. A variety of toxoiding reaction conditions meet expectations.

TABLE 2
Cytoxicity Results for Kinetic Study*

| | Day 0 | Day 1 | Day 2 | Day 3 | Day 4 | Day 5 | Day 6 | Day 7 | Day 9 |
|---|---|---|---|---|---|---|---|---|---|
| Toxoid A, | + | + | + | + | + | + | + | + | + |
| 0.2%, 4° C. | | | | | | | | | |
| Toxoid A, | + | + | + | − | − | − | − | − | − |
| 0.2%, 15° C. | | | | | | | | | |
| Toxoid A, | + | + | − | − | − | − | − | − | N.D. |
| 0.2%, 25° C. | | | | | | | | | |
| Toxoid A, | + | + | + | − | − | − | − | − | − |
| 0.4%, 4° C. | | | | | | | | | |
| Toxoid A, | + | − | − | − | − | − | − | − | − |
| 0.4%, 15° C. | | | | | | | | | |
| Toxoid A, | + | − | − | − | − | − | − | − | N.D. |
| 0.4%, 25° C. | | | | | | | | | |
| Toxoid B, | + | + | + | + | + | + | + | + | − |
| 0.8%, 4° C. | | | | | | | | | |
| Toxoid B, | + | + | − | − | − | − | − | − | − |
| 0.8%, 15° C. | | | | | | | | | |
| Toxoid B, | + | − | − | − | − | − | − | − | − |
| 0.8%, 25° C. | | | | | | | | | |
| Toxoid B, | + | + | + | + | + | + | + | + | + |
| 0.4%, 4° C. | | | | | | | | | |
| Toxoid B, | + | + | + | + | + | − | − | − | − |
| 0.4%, 15° C. | | | | | | | | | |
| Toxoid B, | + | + | − | − | − | − | − | − | − |
| 0.4%, 25° C. | | | | | | | | | |

*+: Cytotoxic; −: No cytotoxicity detected; N.D.: not determined

Kinetic AEX-HPLC Analysis of DoE Reactions

AEX-HPLC (extended gradient method) can be used as a tool to further evaluate the different toxoiding parameters. The AEX profile can be a valuable tool in narrowing down suitable toxoiding conditions. Two subpopulations are observed for both Toxoid A & Toxoid B in the AEX chromatogram both having longer retention times than the toxin. The populations of the peaks shift as the reaction progresses suggesting further modification to the toxin. Potentially, this reflects the formaldehyde reacting with amine groups on the toxin changing the charge characteristics on the protein to be less positive, thereby increasing the binding affinity with the column resin (quaternary ammonium resin). Temperature and Formaldehyde concentration can influence and “shift” the peak population profile as a function of time indicating more formaldehyde protein modification; for both Toxin A and Toxin B toxoiding reactions, a more rapid shift to the second peak population is observed with an increase temperature and formaldehyde concentration. From an evaluation standpoint, it would be more desirable to have a mono-dispersed profile at the second peak position to ensure more protein modification. For Toxoid A, conditions with 0.21% formaldehyde at 25° C., >6 days or 0.42% formaldehyde 15° C., >6 days gave the desired mono-dispersed 2nd peak profile. For Toxoid B, conditions with 0.4% or 0.8% formaldehyde at 15° C. for >10 days; or, 0.4% formaldehyde at 25° C. for >5 days resulted in the desired mono-dispersed 2nd peak profile. It is important to note that reactions with the highest formaldehyde concentrations and temperature began to produce more toxoid populations as a function of time suggesting more extensive protein modification (particularly in the case for Toxoiding A at 0.4% formaldehyde, 25° C.).

Kinetic SEC-HPLC Analysis

The SEC profile can be a valuable tool in narrowing down suitable toxoiding conditions. The chromatograms can give insights into the extent of multimerization that may occur as a result of formaldehyde induced intermolecular crosslinks. It is desired to minimize that amount of multimerization on the toxoids and achieve a profile similar to that with the product produced in Example 1. Individual reactions were monitored daily by SEC-MALS and qualitatively analyzed for the appearance of multimerization. All of the conditions analyzed for the Toxoid B reactions showed no multimerization. For Toxoid A excessive multimerization was observed mainly for the conditions with the highest formaldehyde concentration. Thus the SEC-MALS data does not discriminate for Toxoid B conditions with respect to temperature, time or formaldehyde concentration. However, the data suggests that higher temperature and formaldehyde concentration together can lead to multimerization for Toxoid A.

Kinetic Amine Content (TNBS) Analysis

Formalin mediated toxoiding results in the reduction of free amine content on the protein (e.g., the ε-amino groups of lysine) through reaction to form formaldehyde based moieties. Attempts to monitor the extent of modification using a Trinitrobenzene sulfonic acid (TNBS) assay on the earlier material were made and the extent of modification at the end of the reaction was shown to be ˜35% and 65% for Toxoids A and B respectively (inverse of free amine content remaining). For this study, free amine content was also monitored using TNBS assay. The conditions show that as temperature and time are increased the % free amine content approaches an asymptote more rapidly. Thus the extent of amine modification can be maximally estimated ˜40% for Toxin A and 75% Toxin B (inverse of free amine content remaining). Although the amine content has little correlation with loss in cytotoxicity, it can be used to track the extent of reaction with formaldehyde and the toxins. For examples the amine modification appears to be complete with in 6 days with respect to A and ˜10 days with respect to B at 25° C. If the reaction is performed at lower temperatures, the time taken to achieve the same extent of amine modification increases. Thus data suggests that higher temperatures would lead to a more complete reaction in a shorter amount of time.

Analysis of Antigenicity

An enzyme-linked immunosorbent assay (ELISA) can also be used as a tool to further evaluate the different toxoiding parameters. The ELISA profile of the product can be used to narrow down suitable toxoiding conditions. Toxoids generated were measured via ELISA against antibodies generated from the earlier material and analyzed as a function of toxoiding time. Here ELISA was used to detect the amount of toxin and compared against the concentration measured using absorbance at 280 nm. As the antigen progresses in the toxoiding reaction the ELISA value may drop off indicating a change from response observed with the Example 1 toxoids (potentially indicating multimerization). Although variability was noted in the assay, data suggests that higher temps and higher formaldehyde concentration lead to lower ELISA response. For example, the use of 0.4% formaldehyde at 25° C. results in ELISA values that fall faster than 0.2% formaldehyde at 25° C. Likewise, conditions with 0.4% formaldehyde, 25° C. results in ELISA values that fall faster than those at 0.4% formaldehyde at 4° C. As an evaluation tool, it was desired to keep the ELISA response above 70%; numerous conditions were identified.

Analysis of Immunogencity

Measurement of immunogenicity by hamster potency assay may be used to evaluate the toxoiding conditions. Current specifications set out not less than 4.8 mean Log 10 IgG titer response for Toxoid A and Toxoid B. Toxoids generated from these studies were evaluated according to those specifications and further scrutinized as not having a significantly lower response from toxoids derived from the earlier conditions. Additionally, as all possible permeations (with respect to time, temperature and formaldehyde concentration) could not be evaluated, toxoids were selected based on kinetic cytotoxicity analysis (3× safety margin) as well as physiochemical characteristics described herein. The toxoids were formulated as bivalent material (non-lyophilized) for the hamster potency assay and the sera was analyzed for IgG response. All toxoiding conditions not only passed the potency specification (mean IgG titer response of 4.8 Log 10) but also had statistically equivalent titer response to the earlier (Example 1) material (no significant differences noted). Additionally, all of the sera was tested (using an in vitro challenge assay) and found to have neutralizing antibody activity. As a critical quality attribute, the data suggests that any of these toxoiding conditions could be acceptable.

Reversion Analysis of Drug Product (“DP”)

Drug products (compositions comprising Toxoids A and B) were formulated using the Toxoids A and B prepared using the toxoiding conditions under evaluation. Formulations included either 0%, 0.004%, and in some cases 0.008% (w/v) residual formaldehyde. The formulations were prepared by removing all (or essentially all) of the formaldehyde from Toxoid A or B compositions and then spiking the cleared compositions with the indicated amounts of formaldehyde. The drug products were subjected to a reversion analysis conducted at 37° C. Data from the drug product reversion analysis is portrayed in Table 3. Drug products that tested negative for cytotoxicity are noted (−).

A number of drug product formulations passed the reversion analysis (i.e., had no detectable cytotoxicity following storage at 37° C.). Two drug products (with 0.004% or with 0.008% formaldehyde (“residual formaldehyde”)) had no detectable cytotoxicity following storage at 37° C.: (i) the drug product comprising Toxoid A inactivated by incubation 13 days, 0.2% formaldehyde, 15° C. and Toxoid B inactivated by incubation 13 days, 0.8% formaldehyde, 15° C. (Table 3, parameters of tests 13 and 14); and, (ii) the drug product comprising Toxoid A inactivated by incubation for 6 days 0.2% formaldehyde, 25° C., Toxoid B inactivated for 13 days 0.4% formaldehyde, at 25° C. (Table 3, parameters of tests 22 and 23). Several other drug products with 0.008% formaldehyde also had no detectable cytotoxicity following storage at 37° C. including for example, the drug product comprising Toxoid A inactivated by incubation for 13 days, 0.4% formaldehyde, 4° C. and Toxoid B inactivated for 21 days, 0.8%, 4° C. and the drug product comprising Toxoid A inactivated for 13 days, 0.4% formaldehyde, 4° C. and Toxoid B inactivated for 21 days, 0.8% formaldehyde and 4° C. Optimal toxoiding conditions identified from this analysis were: toxoiding of Toxin A: 0.5 mg/ml Toxin A, 0.21% formaldehyde, 25° C. in 100 mM NaPO₄ pH 7 for 6 days; and toxoiding of Toxin B: 0.5 mg/ml Toxin B, 0.42% formaldehyde, 25° C. in 100 mM NaPO₄ pH 7 for 13 days (Table 3, parameters of test 22). These conditions also had desirable profiles when measured by other physiochemical assays. AEX showed homogenous peak population for each toxoid, SEC MALS showed minimal multimerization and TNBS showed each reaction achieving maximal amine modification at the given time point. Additionally, the ELISA (A280) responses were maintained.

TABLE 3
Reversion Analysis (37° C.)

| | | | 37° C. | | | | | | |
|---|---|---|---|---|---|---|---|---|---|
| Test | Txd A | Txd B | Sample | Week 1 | Week 2 | Week 3 | Week 4 | Week 5 | Week 6 |
| 1 | 6 d, | 21 d, | DP + 0% Form. | + | + | N.D. | N.D. | N.D. | N.D. |
| 2 | 0.4%, | 0.8%, | DP + 0.004% Form. | + | + | N.D. | N.D. | N.D. | N.D. |
| | 4° C. | 4° C. | | | | | | | |
| 3 | 13 d, | 21 d, | DP + 0% Form. | + | + | + | + | + | + |
| 4 | 0.4%, | 0.8%, | DP + 0.004% Form. | + | + | − | − | − | − |
| 5 | 4° C. | 4° C. | DP + 0.008% Form. | − | − | − | − | − | − |
| 6 | 6 d, | 13 d, | DP + 0% Form. | + | + | + | + | + | + |
| 7 | 0.2%, | 0.8%, | DP + 0.004% Form. | + | + | − | − | − | − |
| | 15° C. | 15° C. | | | | | | | |
| 8 | 6 d, | 13 d, | DP + 0% Form. | + | + | N.D. | N.D. | N.D. | N.D. |
| 9 | 0.2%, | 0.4%, | DP + 0.004% Form. | + | + | N.D. | N.D. | N.D. | N.D. |
| | 15° C. | 15° C. | | | | | | | |
| 10 | 6 d, | 18 d, | DP + 0% Form. | + | + | N.D. | N.D. | N.D. | N.D. |
| 11 | 0.2%, | 0.4%, | DP + 0.004% Form. | + | + | N.D. | N.D. | N.D. | N.D. |
| | 15° C. | 15° C. | | | | | | | |
| 12 | 13 d, | 13 d, | DP + 0% Form. | + | + | + | + | + | + |
| 13 | 0.2%, | 0.8%, | DP + 0.004% Form. | − | − | − | − | − | − |
| 14 | 15° C. | 15° C. | DP + 0.008% Form. | − | − | − | − | − | − |
| 15 | 13 d, | 13 d, | DP + 0% Form. | + | + | N.D. | N.D. | N.D. | N.D. |
| 16 | 0.2%, | 0.4%, | DP + 0.004% Form. | + | + | N.D. | N.D. | N.D. | N.D. |
| | 15° C. | 15° C. | | | | | | | |
| 17 | 13 d, | 18 d, | DP + 0% Form. | + | + | N.D. | N.D. | N.D. | N.D. |
| 18 | 0.2%, | 0.4%, | DP + 0.004% Form. | + | + | N.D. | N.D. | N.D. | N.D. |
| | 15° C. | 15° C. | | | | | | | |
| 19 | 6 d, | 6 d, | DP + 0% Form. | + | + | N.D. | N.D. | N.D. | N.D. |
| 20 | 0.2%, | 0.4%, | DP + 0.004% Form. | − | + | N.D. | N.D. | N.D. | N.D. |
| | 25° C. | 25° C. | | | | | | | |
| 21 | 6 d, | 13 d, | DP + 0% Form. | − | + | + | + | + | + |
| 22 | 0.2%, | 0.4%, | DP + 0.004% Form. | − | − | − | − | − | − |
| 23 | 25° C. | 25° C. | DP + 0.008% Form. | − | − | − | − | − | − |

Week 23 DP

| | | | | | | | | | |
|---|---|---|---|---|---|---|---|---|---|
| 24 | 6 d, | 21 d, | DP + 0% Form. | + | + | N.D. | N.D. | N.D. | N.D. |
| 25 | 0.4%, | 0.8%, | DP + 0.004% Form. | + | + | N.D. | N.D. | N.D. | N.D. |
| | 4° C. | 4° C. | | | | | | | |
| 26 | 13 d, | 21 d, | DP + 0% Form. | + | + | + | + | + | + |
| 27 | 0.4%, | 0.8%, | DP + 0.004% Form. | + | + | + | + | − | − |
| 28 | 4° C. | 4° C. | DP + 0.008% Form. | − | − | − | − | − | − |

DP = drug product;
Form. = formaldehyde;
+: Cytotoxic;
−: No cytotoxicity detected;
N.D.: not determined

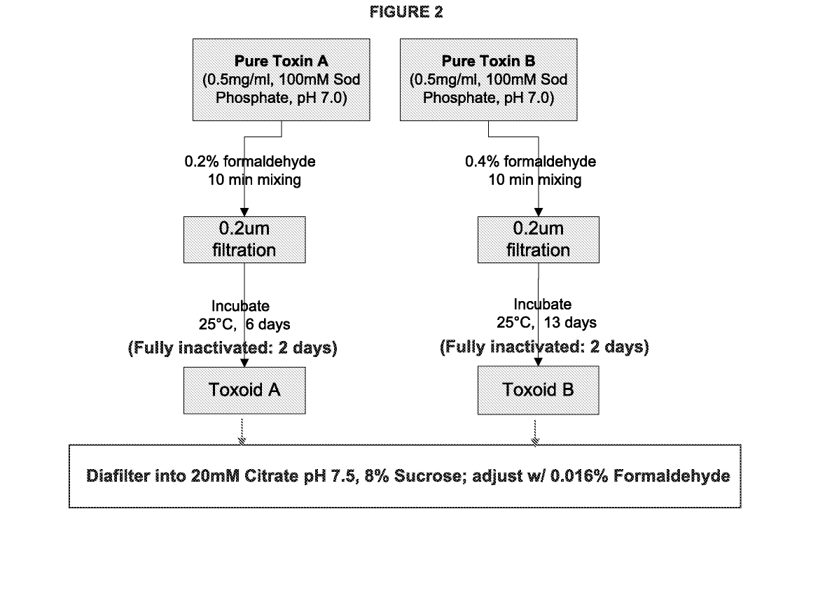

Tables 1 and 3 indicate that the parameters 22 are optimal for preparing toxoids from Toxins A and B. These conditions are:
Preparation of toxoid A: 0.5 mg/ml Toxin A, 0.21% formaldehyde, 25° C. in 100 mM NaPO₄, pH 7 for six days; and,
Preparation of toxoid B: 0.5 mg/ml Toxin B, 0.42% formaldehyde, 25° C. in 100 mM NaPO₄, pH 7 for 13 days.
   These procedures also included a ten minute mixing step followed by 0.2 μm filtration prior to the six day (Toxoid A) or 13 day (Toxoid B) incubation. Prior to testing for reversion at 37° C., Toxoid A and toxoid B were diafiltered into 20 mM citrate, pH 7.5, 0.004% formaldehyde. This procedure is illustrated in FIG. 2. It is also noted that 0.008% formaldehyde also typically provides good stability at 37° C.

This data is further confirmed by surprising immunological data (IgG response in hamsters) showing that longer incubation times resulted in lower ELISA values for Toxoid A, suggesting increased formadelhyde-induced toxin modification (ELISA/A280 at day 6=0.94; at day 12=0.64). In contrast, longer incubation times resulted in higher ELISA values for Toxoid B (ELISA/A280 at day 13=0.53; at day 20=0.73). Desirable ELISA/A280 values are those closer to 1.0. Those of ordinary skill in the art should understand that a 12 day incubation period for toxoiding Toxin A may be appropriate and a 20 day incubation may be appropriate for toxoiding of Toxin A. However, even in view of this data, a 13 day incubation time was considered optimal for toxoiding Toxin B as described above.

Scale Analysis

Toxoids were produced at a larger scale ( 1/10^{th} launch scale (200 L fermentation)) using the optimal toxoiding conditions identified; that is, Toxin A and Toxin B were inactivated using the following conditions: Toxoiding of A: 0.5 mg/mL toxin A, 0.21% (w/v) formaldehyde, 25° C. in 100 mM NaPO₄ pH 7 for 6 days; and, Toxoiding of B: 0.5 mg/mL toxin B, 0.42% (w/v) formaldehyde, 25° C. in 100 mM NaPO₄ pH 7 for 13 days. The kinetics of the toxoiding reaction was evaluated using toxoid samples taken at various time periods during the reaction. In comparison to the toxoids produced at small scale, the toxoids had an identical kinetic cytotoxicity profile, with a loss of cytotoxicity being observed on the 2^{nd} day of the reaction. In addition the toxoids had a similar AEX profile and similar amine modification (as measured by TNBS assay) to toxoids produced at small scale. The immunogenicity of the toxoids generated from the 1/10^{th} scale toxoiding reaction were also evaluated by the hamster potency assay. Like the toxoids produced at small scale, the toxoids gave a mean IgG titer response of 4.8 Log or higher and provided a titer response that was statistically equivalent to that of toxoids prepared in accordance to the process as set out in Example 1. Reversion analysis was conducted on drug product derived from 1/10^{th} scale toxoids and compared to drug product derived from identical toxoiding conditions at small scale. The drug product derived from toxoids at 1/10^{th} scale had the same reversion characteristics as those derived. at the small scale and passed reversion even at 0.004% formaldehyde. Similar results were obtained with Toxoids produced at larger scales (e.g., using 1000 L and 2000 L fermentation cultures). The data from these studies show that the toxoiding method is scalable. The toxoids produced at large scale have identical kinetic cytotoxic profiles, hamster potency and reversion characteristics as those produced at small scale. In regards to reproducibility, the toxoiding process for Toxin A and Toxin B was reproduced more than 6. times and analysis showed similar lot to lot characteristics.

Immunization Studies

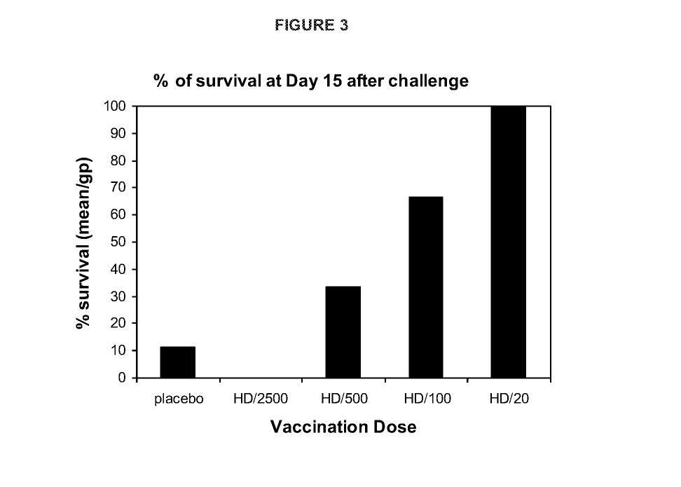

Purified C. difficile Toxoid A and C, difficile Toxoid B were prepared substantially in accordance with the methods described above (e.g., parameters 22 in Table 3) and formulated as vaccine. compositions. Toxoids A and B were combined at a ratio of 3:2 by weight, formulated with a citrate buffer comprising sucrose (4.0% to 6.0% w/v) and formaldehyde (0.012% to 0.020% w/v) and lyophilized. Each composition was reconstituted with diluent as described below and adjuvanted with aluminum hydroxide prior to use as a vaccine for evaluation in the hamster challenge model. Syrian gold hamsters provide a stringent model for C. difficile vaccine development. After being pretreated with a single intraperitoneal (IP) dose of clindamycin antibiotic and after receiving an intragastric (IG) inoculation of toxigenic C, difficile organisms, the hamsters rapidly develop fulminant diarrhea and hemorrhagic cecitis and die within two to four days (e.g., without vaccination). The vaccine was reconstituted with diluent (comprising 0.57% sodium chloride and 800 μg/mL aluminum hydroxide). Serial dilutions of the reconstituted vaccine were prepared. As a human dose (HD) of the vaccine contains 100 μg/dose toxoids and 400 μg/dose aluminum hydroxide, the first dilution prepared, a 1/20 HD contained 5 μg/dose toxoids, 0.008% formaldehyde and 20 μg/dose aluminum. Hamsters (9 hamsters/group) were vaccinated by three intramuscular immunizations (at Day 0, Day 14, and Day 28) with different doses of C. difficile vaccine (4 dilutions of human dose (100 μg/dose) (HD) or were injected with the placebo (AIOOH). At Day 41, hamsters were pretreated with chemical form of Clindamycin-2-phosphate antibiotic at 10 mg/kg by IP route. At Day 42, after 28 hours pretreatment with antibiotic, hamsters were challenged by IG route with a lethal dose of spore preparation derived from C. difficile ATCC43255 strain. The protective efficacy was assessed by measuring the kinetics of apparition of symptoms associated with C. difficile infection and lethality. Results (set out in FIG. 3) demonstrated that the vaccine protects hamsters against lethal challenge with C. difficile toxigenic bacteria in a dose-dependent manner, with 100% protection induced by vaccination with the dose HD/20 (5 μg Toxoid A+B in presence of 20 μg AIOOH). Immunized animals were protected against death and disease (weight loss and diarrhea). The results of this study are representative of several in vivo studies. Accordingly, toxoids prepared by the methods described herein provide protective immunity against C. difficile disease.

### Example 3

### In Vitro Immunization Studies

Toxoids contained in the vaccine formulation were purified from Toxinotype 0. In order to demonstrate that anti-toxin antibodies can neutralize toxin activities from other prevalent variant strains, an in vitro cross-neutralization study was conducted using sera from vaccinated hamsters.

A. Materials and Methods

C. difficile toxoid vaccine was a formalin-inactivated, highly purified preparation of toxoids A and B from C. difficile reference strain VP110463 (ATCC43255), and presented as a freeze-dried preparation that was reconstituted with diluent and mixed with aluminum hydroxide adjuvant (as described above). Placebo was 0.9% saline. Purified native toxins A and B from 087 PCR-ribotype were produced internally as described above. Purified native toxins A and B from 001, 002, 014, 106, 027, 023 and 078 PCR-ribotypes and purified native toxin B from 017 PCR-ribotypes were purchased from TgcBiomics (Bingen, Germany).

Strains VP110463, 630, BAA-1875, ATCC43598 were purchased from ATCC. Strain IPP40348 isolated from France in 2007, was obtained from M. Popoff (Pasteur Institute, Paris France). Strain CDC13695#7, isolated from Canada in 2005, and was obtained from the Center for Disease Control (CDC). Strain SP041, isolated from the US in 2011, and was obtained from D. Gerding. Clinical isolates of C. difficile were obtained from D. Gerding (US and Argentina), F. Barbut (Europe), P. Vanhems (France) and T. Riley (Asia-Pac). C. difficile strains were grown anaerobically in Soy Yeast extract Salt (SYS) medium for 16 hours then expanded on SYS medium supplemented with sorbitol for 72 hours. Bacterial culture supernatants were then harvested, filtered and supplemented with anti-proteases and 30% glycerol.

Quantification of toxins A and B present in the bacterial culture supernatants was performed using a commercial ELISA method (tgcBIOMICS GmbH, Mainz, Germany) according to the manufacturer's instructions. Briefly, microtitre plates coated with capture antibodies to both toxin A and toxin B were incubated with culture supernatants or standard control toxins for 60 min at 37° C. Following washing of the unbound material, specific monoclonal antibody to either toxin A or toxin B (conjugated to horseradish peroxidase) was added to wells; microtitre plates were incubated for 60 min at 37° C. Subsequent to a second wash step, substrate was added to allow colour development at room temperature for 30 min. The reaction was stopped by addition of H₂SO₄ to each well, and the ELISA was analysed by a spectrophotometer at 450 and 620 nm.

A bioassay was developed for the in vitro cross-neutralization analysis. It is adapted from the well-known IMR90 Toxin Neutralizing Assay. The design of the bioassay consisted of pre-incubating a predetermined dilution of either hamster serum raised against C. Difficile Toxoid Vaccine known to neutralize the cytotoxic activity of purified toxins from the vaccine strain or placebo serum with serial dilutions of either purified native toxins A or B from clinically-relevant C. difficile prototype strains or filtered bacterial supernatants from prototype strains or clinical isolates, containing toxins A and/or B. The serum-toxin mixture was added onto IMR-90 cells, which were pre-seeded the day before onto Eplates to reach confluence and attachment on the electrodes. Plates were then incubated at 37° C. E-Plates are specially-designed tissue culture microtiter plates containing interdigitated gold microelectrodes on the bottom. They are intended to be used with the xCELLigence system to noninvasively monitor cellular events in real time, using electrical impedance as the readout, without the incorporation of labels. IMR90 cell rounding induced by toxin will lead to a decrease in electrode impedance, which is displayed as cell index (CI) values. Corresponding vaccine- and placebo-cell indexes were then plotted as cytotoxic curves and modelled by sigmoid response curves (four-parameters logistic) using in-house software. The bacterial supernatant dilution for each clinical isolate inducing 50% cytotoxicity, defined as IC50, was determined. The shift between both curves at 50% cytotoxicity was calculated as the Relative Efficacy (RE). The RE represents the capacity of vaccine specific anti-toxin antibodies to neutralize the toxin-cytotoxic activity of either purified toxins or clinical isolates. The threshold from which RE was considered statistically significant was established by the determination of the intermediate precision using both specific anti-toxin antibodies and irrelevant antibodies against placebo and was therefore defined as 5.4.

B. Results

The efficacy of vaccine serum was first tested against purified native toxins A and B from the prevalent toxinotypes. Included in this test were toxins from the reference strain VP110463 (ATCC43225 PCR-Ribotype 087) and from strains of PCR-Ribotype 001, 002, 014, and 023, 017 (A⁻B⁺CDT⁻), as well as from the so-called hypervirulent PCR-Ribotypes 027 (A⁺B⁺CDT⁺) and 078 (A⁺B⁺CDT⁺). The concentration of each purified native toxins at which 50% cytotoxicity is achieved was first evaluated. IMR-90 cells are fairly sensitive to purified toxins A and B from the different PCR-Ribotypes tested. The IC50 concentration for toxins A was in a similar range, ranging from 4.4 ng/mL to 15.9 ng/mL. In contrast for toxins B, the IC50 concentration varied depending on the selected PCR-ribotypes and ranged between 0.2 ng/mL to 6.2 μg/mL suggesting different potencies among toxin B. The relative efficacy (RE) of vaccine specific anti-toxin antibodies to neutralize the respective cytotoxic activity of each purified toxin was then evaluated as described in Materials and Methods. As shown in Table 4, the calculated REs ranged from 9.3 to 818.5, which were all above the positive threshold defined as RE=5.4. The relative efficacy of vaccine sera to neutralize purified toxins from PCR-Ribotypes 027, 023 and 078 was lower compared to other PCR-ribotypes but still above threshold. This result suggests vaccine sera were able to significantly neutralize cytotoxicity of all tested purified toxins.

TABLE 4
Neutralization of cytotoxic activity of purified toxins from prevalent toxinotypes

| | | Purified native toxins | | | | | |
|---|---|---|---|---|---|---|---|
| | | Toxin A | | | Toxin B | | |
| | | IC50^{a} (ng/mL) | | | IC50 (ng/mL) | | |
| | PCR- | Placebo | Vaccine | | Placebo | | |
| Toxinotype | ribotype | serum | serum | RE^{c} | serum | Vaccine serum | RE |
| 0 | 087 | 4.4^{b} | 230.9^{b} | 52.3^{b} | 0.2^{b} | 7.3^{b} | 38.1^{b} |
| | 001 | 15.9 | 388.0 | 24.4 | 108.0 | 88 400.0 | 818.5 |
| | 002 | 5.8 | 256.0 | 44.4 | 98.1 | 38 000.0 | 387.4 |
| | 014 | 8.9 | 609.0 | 68.3 | 6 220.0 | 59 4000.0 | 95.7 |
| III | 027 | 5.4 | 95.3 | 17.8 | 3.5 | 32.2 | 9.3 |
| IV | 023 | 9.4 | 145.0 | 15.4 | n.d. | n.d. | n.d. |
| V | 078 | 6.9 | 97.5 | 14.1 | 4.5 | 58.8 | 13.1 |
| VIII | 017 | n.d.^{d} | n.d. | n.d. | 17.1 | 2 560.0 | 149.7 |

^{a}IC50 = concentration inducing 50% cytotoxicity
^{b}Geometric mean of 3 independent experiments
^{c}RE = Relative Efficacy, considered statistically significant if above threshold 5.4
^{d}n.d. = not determined

In addition to purified native toxins, it was also important to evaluate the relative efficacy of vaccine specific anti-toxin antibodies to neutralize toxins produce by C. difficile prototype strains representative of the most prevalent strains, since the majority of circulating pathogenic strains expresses both toxins. To this end, several prototype strains (Table 5) were selected based on the most prevalent types. The concentration of toxin A and toxin B present in the culture supernatant were quantified by ELISA and the dilution of bacterial supernatant inducing 50% toxicity on IMR-90 cells was calculated (IC50) for each prototype strain. Strain ATCC®43255™ (VP110463), known to be a high toxin-producer strain in optimal culture condition, produced high level of toxin A (17.04 μg/mL) and toxin B (7.06 μg/mL) and was very potent in inducing toxicity, with a IC50 dilution of placebo serum at 5.6×10⁷. In comparison, the other strains produced low to barely undetectable level of toxins. Toxicity was proportional to the quantity of toxin present in the supernatant, since the strains were still able to induce toxicity with a lower IC50 dilution of placebo serum, ranging from 4.4×10³ to 5.2×10⁶. Surprisingly, despite a various range in the toxicity potency (IC50), vaccine-serum was able to neutralize cytotoxic activity of all bacterial supernatants, since all calculated REs (shift between IC50 dilutions of the placebo serum to the vaccine-serum) were all above significant threshold. Similar to purified toxins, the lowest RE observed was against strains of Toxinotype III PCR-Ribotype 027 and Toxinotype V, PCR-Ribotype 078, but still above significant threshold.

TABLE 5

| | | | Concentration | | IC50^{a} Dilution | | |
|---|---|---|---|---|---|---|---|
| | PCR- | | (μg/mL) | | Placebo | Vaccine | |
| Toxinotype | ribotype | Strain Identification | Toxin A | Toxin B | serum | serum | RE^{b} |
| 0 | 087 | ATCC ®43255 ™ | 17.04 | 7.06 | 5.6 × 10⁷^{c} | 1.6 × 10⁴^{c} | 65.3^{c} |
| | | (VPI10463) | | | | | |
| | 012 | ATCC ®1382 ™ (630) | n.d.^{d} | n.d. | 8.2 × 10⁴ | 1.7 × 10³ | 38.3 |
| | 001 | NCTC11204 | 0.07 | 0.00 | 2.4 × 10⁴ | 6.2 × 10² | 47.1 |
| | | NCTC11209 | 1.71 | 0.09 | 5.4 × 10⁵ | 1.2 × 10⁴ | 43.5 |
| | 002 | NCTC12729 | 0.48 | 0.03 | 8.2 × 10⁴ | 6.2 × 10⁴ | 16.2 |
| III | 027 | ATCC ®BAA-1870 ™ | 1.27 | 2.74 | 5.2 × 10⁶ | 3.2 × 10⁵ | 16.2 |
| | | IPP40348 | 0.91 | 1.72 | 6.1 × 10⁶ | 4.4 × 10⁵ | 13.8 |
| | | 13695#7 (epidemic) | 1.91 | 0.39 | 9.9 × 10⁵ | 7.9 × 10⁴ | 12.5 |
| | | CD196 (historic, non- | 0.80 | 0.99 | 2.2 × 10⁶ | 1.4 × 10⁵ | 14.9 |
| | | epidemic) | | | | | |
| | | R20291 (epidemic) | 0.99 | 0.789 | 1.8 × 10⁶ | 1.3 × 10⁵ | 13.4 |
| IV | 023 | NK91 | 0.08 | 0.00 | 5.2 × 10³ | 2.3 × 10¹ | 230.5 |
| V | 078 | ATCC ®BAA-1875 ™ | 0.02 | 0.00 | 4.7 × 10³ | 6.2 × 10² | 7.6 |
| VIII | 017 | ATCC ®43598 ™ | 0.00 | 0.00 | 4.4 × 10³ | 3.9 × 10¹ | 113.0 |

^{a}IC50 = concentration inducing 50% cytotoxicity
^{b}RE = Relative Efficacy, considered statistically significant if above threshold 5.4
^{c}Geometric mean of 4 independent experiments
^{d}n.d. = Not Determined

In order to ensure a broad representation of prevalent circulating C. Difficile toxin variant strains, the in vitro cross-neutralization study was expanded to a large C. difficile strain collection, with recent circulating clinical isolates and a large panel of isolates analyzed for each Toxinotype. More than 500 recent clinical isolates from prospective clinical and epidemiological studies worldwide were collected (more than 80 clinical isolates were collected in 2011 within the US and Argentina; more than 350 clinical isolates were collected in 2005 throughout Europe and 60 isolates were collected between 2010 & 2011 from France; more than 30 clinical isolates were collected between 2012 and 2013 in Asia-Pacific countries, including Australia, Singapore, Japan, Korea, Indonesia and Taiwan). More than 150 C. difficile worldwide clinical isolates were selected from this collection for the in vitro analysis. The selection was based on multiple criteria such as countries of isolation, molecular typing and, when available, clinical parameters such as CDI severity and CDI episode. The geographical and molecular distribution of clinical isolates is described in Table 6.

TABLE 6
Summary of clinical isolates tested in the in vitro study

| | PCR- | Number of isolates by | | | |
|---|---|---|---|---|---|
| | ribotype | Geographical Origin | | | |
| Toxinotype | (RT) | Europe^{a} | U. States | Argentina | Asia-Pac^{b} |
| 0 | 001 | 5 | 1 | — | — |
| | 002 | 7 | — | — | 4 |
| | 014 | 5 | — | 1 | — |
| | 020 | 4 | — | — | — |
| | 014/020 | — | — | — | 5 |
| | 014/020/077 | 3 | — | — | — |
| | 106 | 2 | 1 | 1 | — |
| | 018 | 1 | — | — | 2 |
| | 053 | | | — | 1 |
| | others | 42 | 4 | — | — |
| I | n.d.^{c} | 1 | 1 | — | — |
| III | 027 | 6 | 4 | — | — |
| | 075 | 1 | — | | — |
| | others | 1 | — | — | — |
| IV | 023 | 4 | — | — | — |
| V | 078 | 4 | 1 | — | — |
| | 079 | 1 | — | — | — |
| | 122 | 1 | — | — | — |
| | 126 | 4 | 1 | — | — |
| | 078/126 | 2 | — | — | — |
| VI | 127 | — | — | — | 2 |
| VI or VII | 66-2 | 1 | — | — | — |
| VIII | 017 | 8 | 1 | 2 | 5 |
| IX | 019 | 1 | — | — | — |
| XII | 056 | — | — | — | 1 |
| | n.d. | 1 | — | — | — |
| others | 046 | — | — | — | 5 |
| | 369 | — | — | — | 3 |

| | | | | |
|---|---|---|---|---|
| Total | 106 | 14 | 4 | 28 |

^{a}Countries of Europe include Belgium, France, Germany, Ireland, Hungary, Italy, Netherlands, Poland, Spain, Switzerland, Sweden, Turkey, Greece, UK
^{b}Countries of Asia-Pac include Japan, Korea, Singapore, Taiwan and Australia
^{c}n.d. = PCR-ribotype unknown

Ten toxinotypes were represented within the different geographical region, including the 5 most prevalent ones, with a majority of toxinotype 0, then toxinotype III, V, VIII and IV, as well as others such as toxinotype I, toxinotype IX and XII. Considering ribotype (RT) distribution, more than 23 ribotypes were represented, with more than 13 RT for the 13 ribotypes for Toxinotype 0. The selection reflects the five lineages/toxinotypes identified as being the most prevalent in CDI patient with the specific distribution of each toxinotype in the different part of the world: for example, in Europe a majority of toxinotype 0 strains, in the US a majority of toxinotype 0 and III strains, and in Asia-Pac a majority of toxinotypes 0 and VIII, as well as other RT 046.

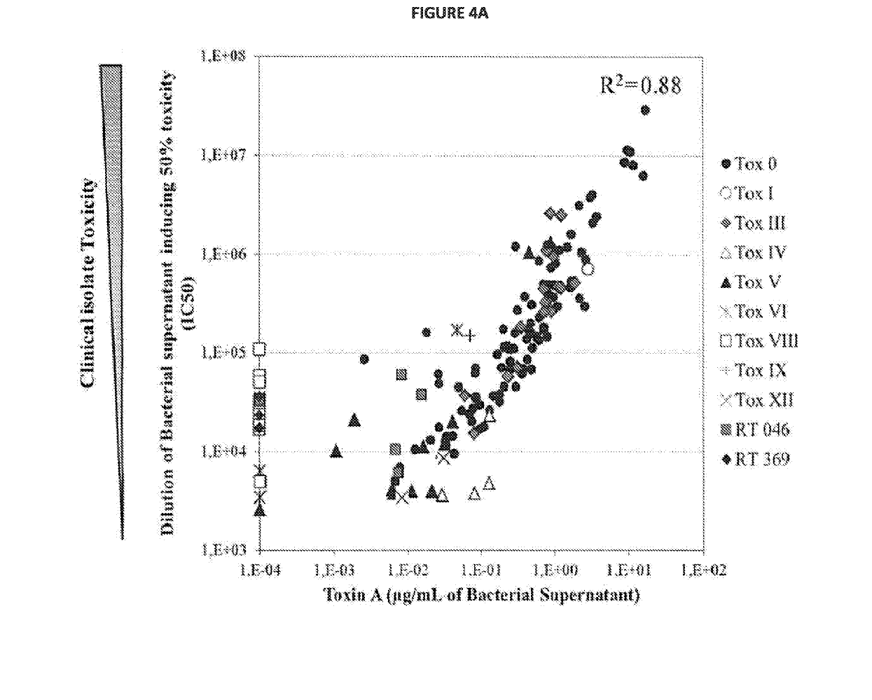

Bacterial supernatant containing toxins were generated from in vitro culture of clinical isolates. Concentration of toxin A and toxin B in culture media were quantified by ELISA for each clinical isolate. The in vitro toxicity of each clinical isolates was also evaluated by calculating the IC50. The IC50 plotted against the level of toxin A and B present in the supernatant for each clinical isolates (FIG. 4). All bacterial supernatants were toxic in the assay with a broad range of toxicity for each toxinotype. The toxicity was strongly proportional to the level of toxins present in the bacterial supernatant, with a correlation coefficient of 0.88 and 0.93 for toxin A and toxin B, respectively (FIG. 4A and FIG. 4B), likely related to the capacity of each clinical isolate to grow in culture (not shown). In general clinical isolates produce more toxin A than toxin B. Toxin A level was undetectable for toxinotype VIII clinical isolates, which are known to only produce toxin B. For some bacterial supernatants, toxin B was below detection limit (1.E+04) under these culture conditions.

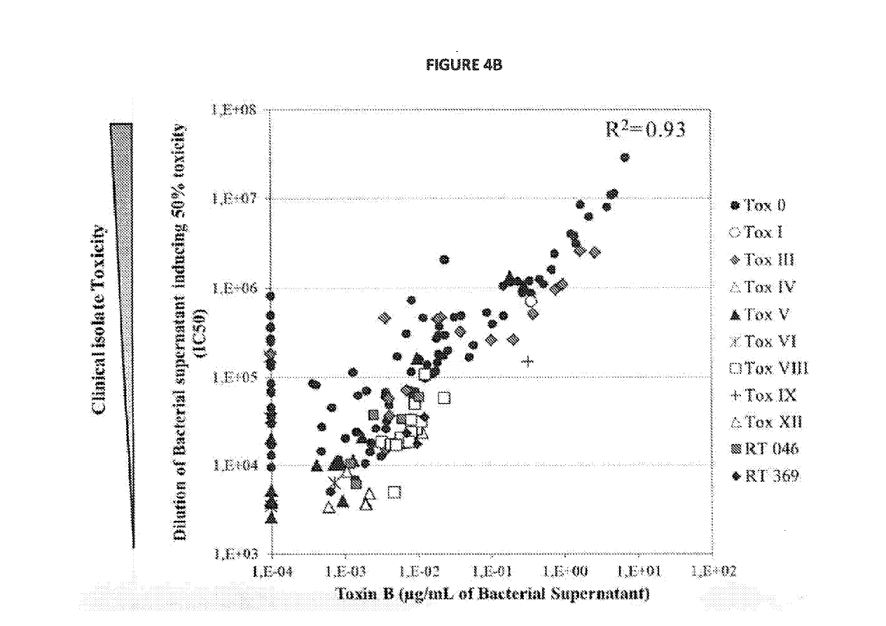

The RE of each clinical isolate was then calculated and plotted against their respective cytotoxic index in order to evaluate potential clusters among toxin variant types (FIG. 5). All bacterial supernatants were neutralized by vaccine-induced serum since the RE was above threshold. Relative Efficacy (RE) was independent of the strain toxicity (IC50). Interestingly, despite a broad range of cytotoxicity among same toxinotypes, a homogeneous RE behavior for clinical isolates from the same toxin-variant types was observed. The RE for Toxinotypes III, V and VI was lower compared to RE for Toxinotype 0, but still above threshold. It was demonstrated this was not related to binary toxin activity, since IMR90 cells were not sensitive to binary toxin (data not shown), but rather consistent with toxin A- and toxin B-phylogeny based on sequence comparison.

This comprehensive study in the hamster model demonstrates broad coverage against the five most prevalent variant strains circulating worldwide, namely Toxinotype/RT 0/012, III/027, IV/023, V/078 and VIII/017. C. difficile Toxoid Vaccine generates anti-toxin antibodies in the hamster model capable of neutralizing in vitro toxin A and B from key toxinotypes 0, III, IV, V, VIII, and others (I, VI, XII). The lower Relative Efficacy (RE) observed for Toxinotypes III, V and VI is not related to binary toxin activity and is consistent with toxin A and toxin B phylogeny. It is important to note that the presence of binary toxin has been associated with a marked increase in disease severity and risk of death. The role of binary toxin is unknown.

### Example 4

### Cross-Immunization Studies

The immunization studies described above with respect to the C. difficile VP110463/ATCC43255 strain in the hamster model with the VPI 1064631 ATCC43255 strain as the challenge strain were performed using different C. difficile strains to determine whether the same could be used to vaccinate animals against multiple strains (i.e., provide cross-protection). Strains having the toxinotypes A⁺B⁺CDT⁻, A⁺B⁺CDT⁺, and A⁻B⁺CDT⁻ were studied, as described below.

The studies used purified C. difficile Toxoid A and C. difficile Toxoid B derived from the same C. difficile strain prepared substantially in accordance with the methods described above (e.g., parameters 22 in Table 3) and formulated as vaccine compositions with aluminum hydroxide (5 μg Toxoid A+B in presence of 20 or 160 μg AIOOH (“C. difficile Toxoid Vaccine”)).

The in vivo cross-protection studies were conducted in the clindamycin-induced lethal enterocolitis Golden Syrian hamster model. This model is indeed commonly used for studying pathogenesis of C. difficile infection and protection mediated by vaccines. Hamsters, immunized with either C. difficile toxoid vaccine or diluent buffer (placebo), were pretreated with Clindamycin-2-phosphate solution, to disrupt the gut microbiota and render the animals susceptible to subsequent lethal challenge with C. difficile spore-enriched preparations from different prototype strains.

Protection against CDI was evaluated by monitoring the onset of clinical signs, including diarrhea, and survival. Prototype strains selected for in vivo cross-protection studies are representative the five most prevalent toxin-variant strains (Table 4).

A. Materials and Methods

Female Golden Syrian hamsters (Mesocricetus auratus) from Charles River Laboratories (Germany), 70-90 g, were used for immunization and challenge studies. Animals were randomly distributed within groups and they were housed at 3 per cage 800 cm² (type 3, ref: LF-3H, supplier Serlab). After C. difficile challenge, animals were housed individually in cages with isocaps. Based on biostatistician analysis, nine animals per group were used. The hamsters were injected three times via the intramascular (IM) route, two weeks apart with either C. difficile toxoid vaccine or aluminum diluent buffer (placebo control). On day 41, 10 mg/kg of Clindamycin-2-phosphate solution was administered via the intraperitoneal (IP) route. Twenty four hours later, hamsters were challenged intragastrically (IG), using a feeding needle, with a predetermined lethal dose of live C. difficile spore-enriched preparations of each prototype strain. Post challenge, animals were observed at least twice a day for morbidity and mortality. Body weight was also monitored at precise time prior to the clindamycin injection and then one to three times per week during the clinical monitoring. Diarrheal disease was reported as a group median score of individual illness scores: 0=no disease; 1=loose feces; 2=wet tail and perianal region; 3=wet perianal region, belly and hind paws; and 4=death. Statistical analysis were done using SAS v9.2® and Excel softwares. Kaplan-Meier method with log-rank test was used for estimation of the survival function from life-time data. Bilateral Fisher exact test was used to compare the percentage of survival animals at Day 17 post challenge. A margin of error of 5% was used for effects of the factors.

C. difficile prototype strains for challenge were anaerobically grown in Thioglycolate medium for 24 hours at 37° C. The culture was then inoculated on anaerobic blood agar plates (CDC, Becton Dickinson) and incubated at 37° C. for 7 days to induce spore formation. Spores were then harvested into PBS without Ca or Mg, washed twice then heat shocked at 57° C. for 10 minutes to kill the vegetative cells. Spore suspension was centrifuged at 500 g for 30 minutes and re-suspended in 20% glycerol in PBS. Spore preparations were frozen at <−70° C. for long term storage. Viable spore counts (CFU/mL) were assessed by thawing the spore stock at 37° C. and performing serial 10-fold dilutions in water. Dilutions were plated in triplicate onto Brain Heart Infusion medium with yeast extract agar plates (BHISA, Becton Dickinson) in presence of 0.1% of taurocholate (Sigma) to enhance spore recovery. Plates were incubated under anaerobic conditions at 37° C. for no less than 48 hours. The colonies were counted and CFU/mL was calculated.

B. Results

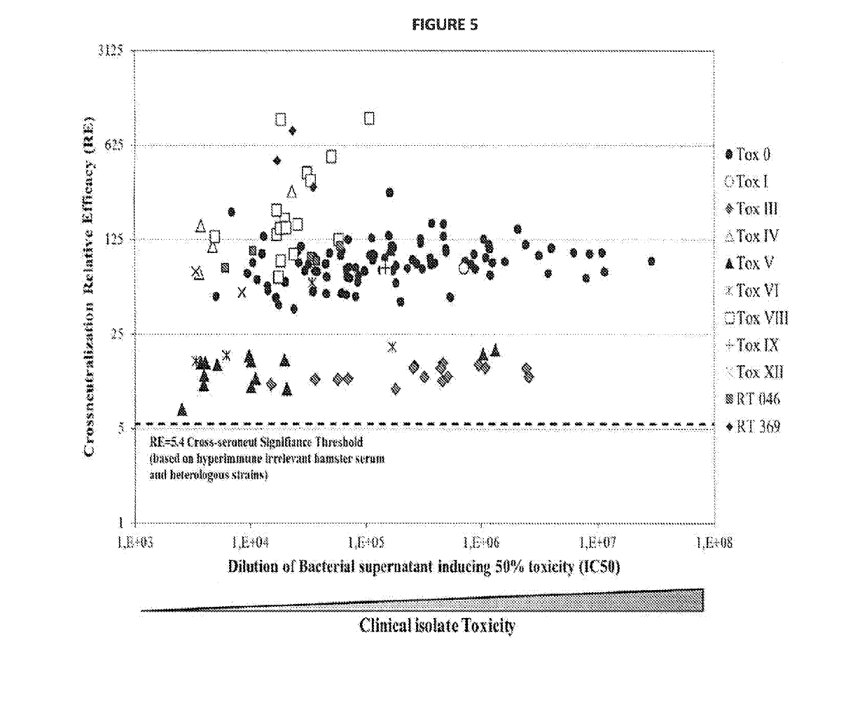

Protection against the homologous C. difficile vaccine strain was demonstrated (FIG. 6). In the placebo group, the onset of acute diarrhea appeared as early as 1 day after challenge (FIG. 6A) and the survival rate dropped rapidly to 11% as early as 3.5 days after challenge (FIG. 6B). In the vaccine group (solid squares), very limited feces change was observed throughout the study, with only one hamster exhibiting transient mild diarrhea (score 2) starting at day 2.5 with recovery at day 9 after challenge and significant protection was observed since survival rate remained at 100% throughout the study.

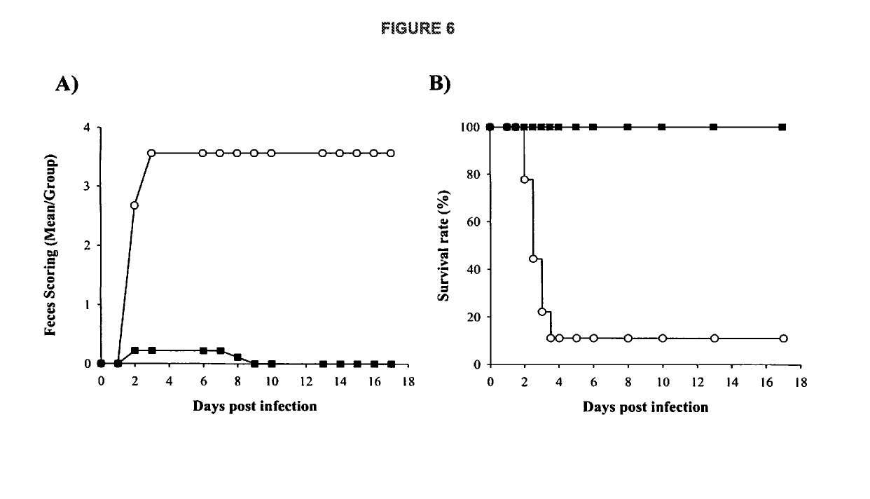

To demonstrate protection against another toxinotype 0 strain from a different RT, C. difficile 630 strain was used (FIG. 7). The onset of diarrhea appeared as early as 3 days after challenge to reach its maximum 6 days after challenge in the placebo group (FIG. 7A). In the vaccine group, very limited feces change was observed throughout the study. The survival curve in FIG. 7B shows that, in the placebo group (open circles), survival dropped as early as 2 days after challenge to reach 11% 13 days after challenge. In the vaccine group (solid squares), survival remained at 100% throughout the study. C. difficile Toxoid Vaccine induced a significant protection against disease and death (p=0.0004 with Fisher exact test and p=0.0001 with the Kaplan Meier log-rank test) after challenge with strain 630.

Protection was also evaluated against hypervirulent fluoroquinolone-resistant toxinotype III PCR-Ribotype 027 strains which are, in addition to A⁺B⁺, express binary toxin (CDT⁺). For this purpose, three strains were selected for the analysis: strain IPP40348 isolated from France in 2007, strain CDC13695#7 isolated from Canada in 2005, and strain SP041 isolated from US in 2011. An acute CDI was induced after challenge with the three strains with acute diarrhea starting as early as two days after challenge and 100% lethality achieved within less than five days after challenge.

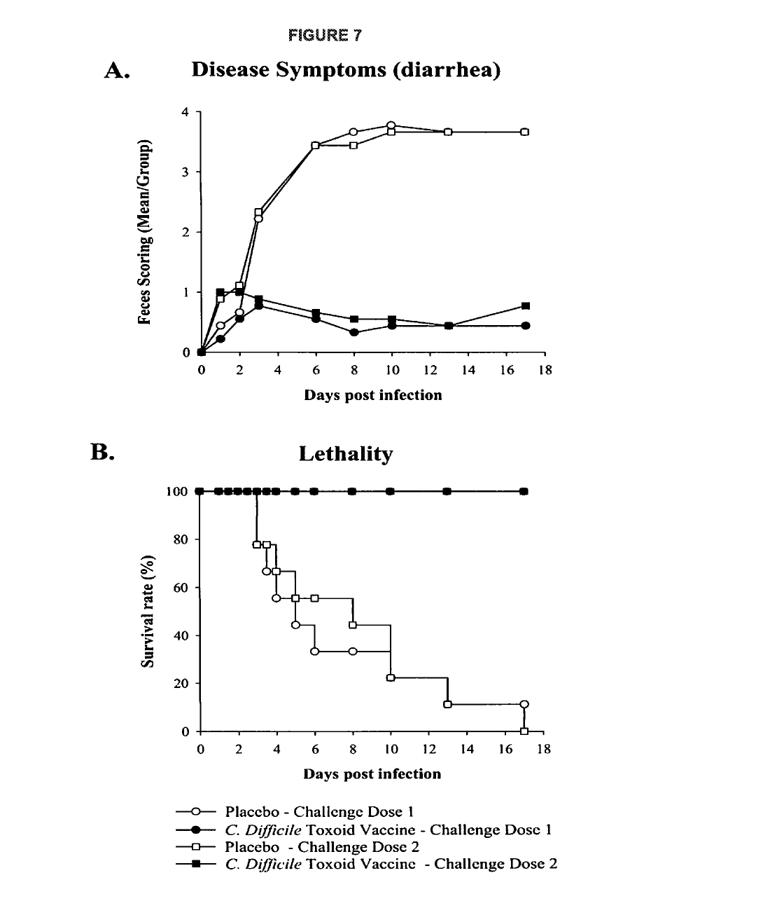

Following administration of the formalin-inactivated, highly purified preparation of toxoids A and B from C. difficile reference strain VP110463 (ATCC43255) described above mixed with 160 μg ALOOH both low symptoms and lethality (FIGS. 8A-B). Most of the hamsters (10/12) vaccinated exhibited no disease symptoms and survived the challenge, providing a significant protection (p-values <0.001 with both Fisher exact and Kaplan Meier log-rank tests). Only two hamsters exhibited moderate diarrhea (score 2, with a mean/group of 0.42) within three days after challenge and succumbed six days after challenge.

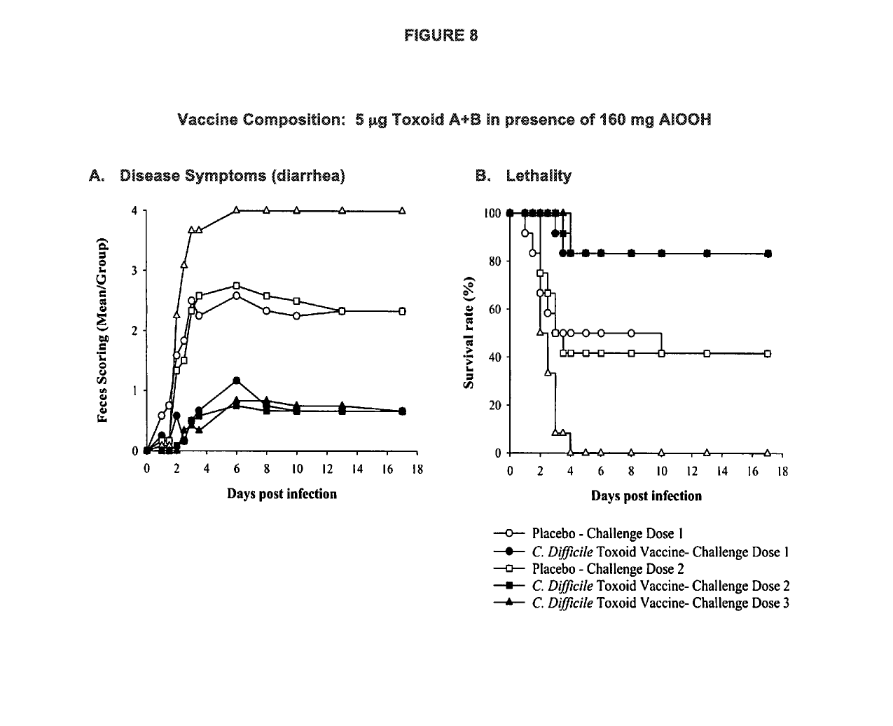

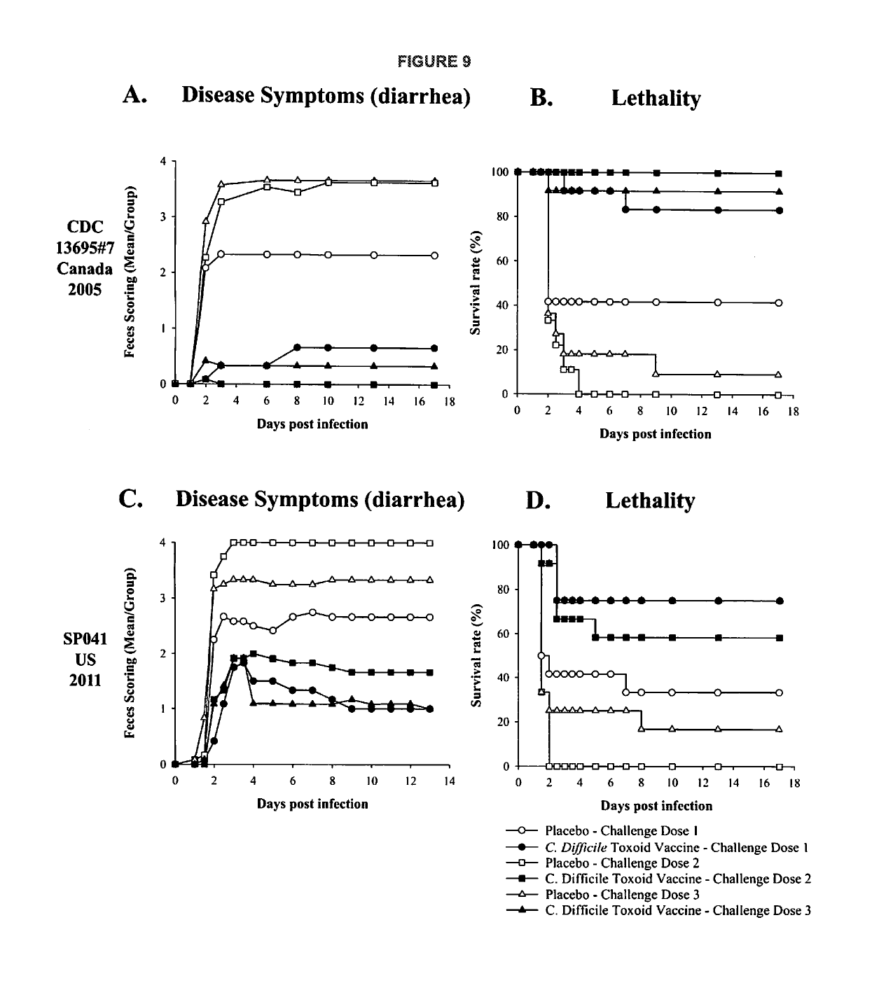

After challenge with strain CDC13695#7, the C. difficile Toxoid Vaccine elicited a significant cross-protection to disease symptoms (FIG. 9A) and death (FIG. 9B) (p-values <0.001 with both Fisher exact and Kaplan Meier log-rank tests). Indeed, none of the vaccinated hamsters exhibited any loose stool and the survival remained at 100% after challenge, whereas in the control groups, hamsters exhibited strong diarrhea with wet perianal region, belly and hind paws (score 3) within 2 days after challenge and the survival rate dropped to 8% after challenge within 4 days after challenge.

For the recent American clinical isolate, strain SP041, a strong and acute diarrhea (score 3) was observed for all the hamsters in the placebo group leading to 100% lethality within 2 days after challenge (FIGS. 9C-D), suggesting a highly virulent strain. Among hamsters vaccinated with the C. difficile Toxoid Vaccine, 58% (7/12) were free of any disease symptoms and survived to the challenge for more than 17 days. The remaining 42% (5/12) exhibited moderate to acute diarrhea and died within 2 to 5 days after challenge. The protection was nevertheless significant (p-value <0.001 with Kaplan Meier log-rank test and p-value=0.005 with Fisher exact test).

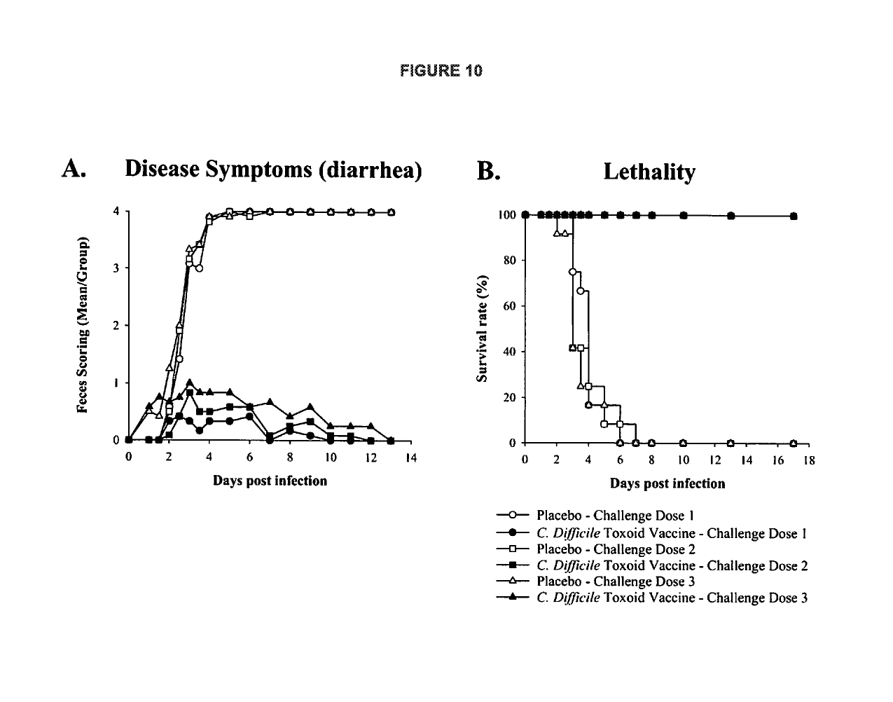

Toxinotype V PCR-ribotype 078 hypervirulent strains are also prevalent worldwide and are A+B+CDT+. Strain BAA-1875 was used as a prototype strain to evaluate cross-protection. In the placebo group (open circles), challenge with the strain led to a strong and acute diarrhea (score 3) observed in 67% hamsters (group mean 1.7) within 2 days after challenge (FIG. 10A) and 100% lethality within 3 days after challenge (FIG. 10B). The C. difficile Toxoid Vaccine induced a significant cross-protection with a survival rate remaining at 100% throughout the study (all p-values ≤0.0001 with both Fisher exact and Kaplan Meier log-rank tests) and absence of any disease symptoms in the vaccinated hamsters.

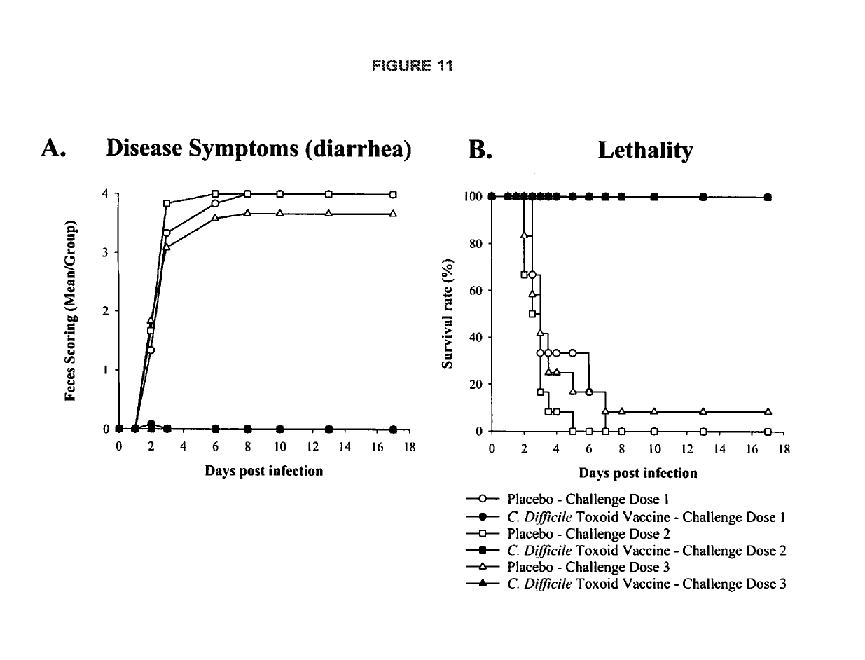

Toxinotype IV PCR-ribotype 023 strains are emerging in different countries. Interestingly the strains are also A⁺B⁺CDT⁺. It was therefore important to evaluate cross-protection against one prototype strain. Strain NK91, recently isolated from a French hospital in 2012 was used as prototype strain (FIG. 11). In the placebo group, most of the hamsters exhibited from loose stool (score 1), as early as one day after challenge, to acute diarrhea (score 3), in less than 3 days after challenge (FIG. 11A) leading to 100% lethality within 6 days after challenge (FIG. 11B). The C. difficile Toxoid Vaccine induced a significant cross-protection with a survival rate remaining at 100% throughout the study (p-values <0.001 with both Fisher exact and Kaplan Meier log-rank tests) with mild and transient diarrhea (score 1, loose stool) resolved within 13 days after challenge.

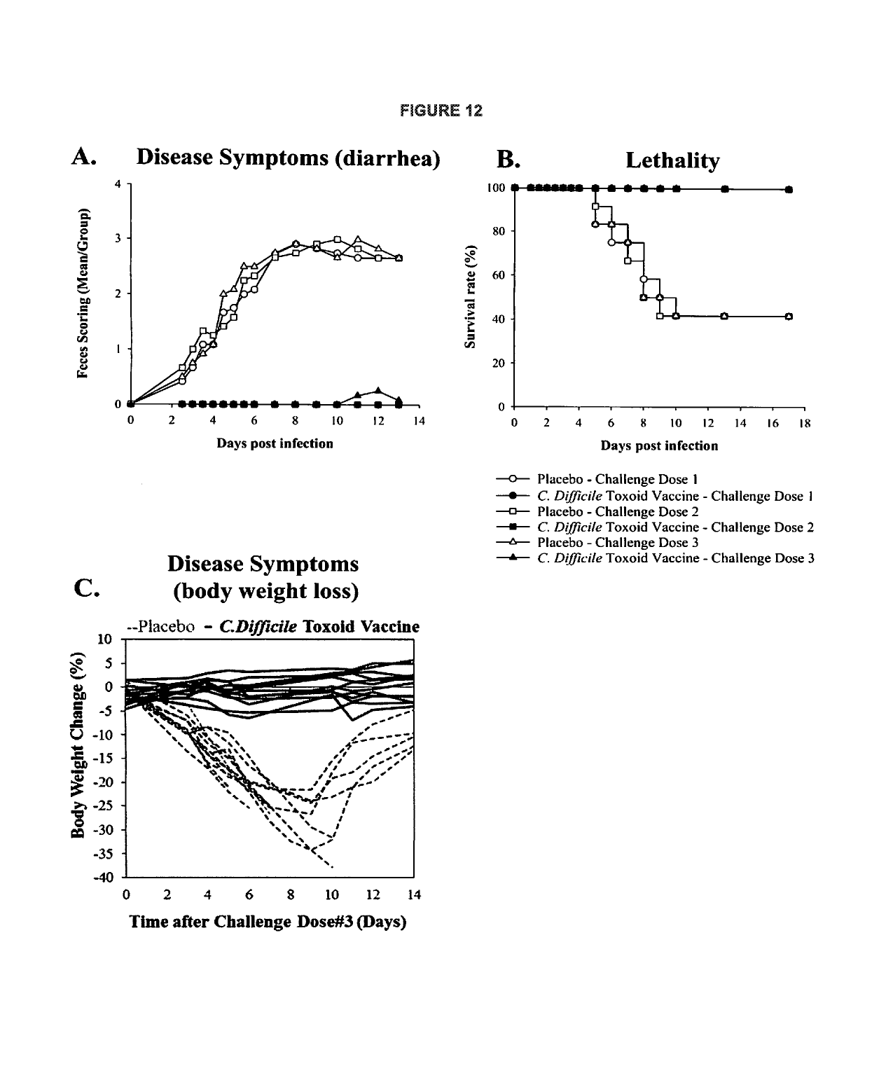

Toxinotype VIII PCR-ribotype 017 strains, which are A⁻B⁺CDT⁻, are highly prevalent in Asia Pacific region. Strain ATCC43598 was used as a prototype strain to evaluate cross-protection (FIG. 12). In the placebo group, challenged hamsters exhibited a strong diarrhea within three to five days after challenge (FIG. 12A) and strong body weight loss that can decrease by 35% within 9 days after challenge (FIG. 12B). Seven out of the 12 sick hamsters succumbed to their symptoms, with a survival rate dropping to 42% within 10 days after challenge (FIG. 12C). Some hamsters were however able to recover from their symptoms. Despite the absence of toxin A, strain ATCC43598 is therefore still virulent, but with a reduced incidence and severity of disease in the hamsters, as already described. Surprisingly, all hamsters vaccinated with the C. difficile Toxoid Vaccine were protected against disease symptoms (FIGS. 12A and 12B) and death (FIG. 12C) and the survival rate remained at 100% after challenge (p-value=0.0046 with a Fisher exact analysis, p-value=0.0020 with Kaplan Meier analysis).

The assayed strains are representative of the five most prevalent toxinotypes: 0, III, IV, V, and VIII. It was surprisingly observed that the composition provided significant cross-protection against symptoms and death after challenge with Toxinotype 0, PCR-ribotype 012, strain 630 (A⁺B⁺CDT⁻); Toxinotype III PCR-ribotype strain 027 strains (A⁺B⁺CDT⁺) (CDC 13695#7 strain, Canada, 2005; SP041 clinical isolate, US, 2011; IPP40348 strain, France, 2007; Toxinotype IV PCR-ribotype 023 (A⁺B⁺CDT⁺) (clinical isolate NK91, France 2012; Toxinotype V PCR-ribotype 078 strain ATCC BAA-1875 (A⁺B⁺CDT⁺) and Toxinotype VIII PCR-ribotype 017 (ATCC4539 (A⁻B⁺CDT⁻). It is noted that cross-protection was not significant in this study for strain IPP40348 using the composition comprising 20 μg aluminum hydroxide (AIOOH) but was significant when 160 μg AIOOH was included. The data is summarized in Table 7.

TABLE 7

| | | | | | | In vivo |
|---|---|---|---|---|---|---|
| | | | | | | crossprotection |
| | | | | Immunization Dose | | % survival vaccine |
| Toxinotype | PCB- | Prototype | | Bivalent | AlOOH | vs placebo |
| Phenotype | ribotype | Strain | Origin Date | Toxoid | Adjuvant | (S if ≠ is significant) |
| 0 | 087 | VPI10483 | Vaccine strain | 5 μg | 160 μg | 100% vs 0% (S) |
| A+B+ CDT− | | (ATCC43255) | ATCC | | or | |
| | | | | | 20 μg | |
| | 012 | 630 | ATCC | 5 μg | 20 μg | 100% vs 0% (S) |
| III | 027 | IPP40348 | France 2007 | 5 μg | 20 μg | 33% vs 0% (NS) |
| A+B+ CDT+ | | | (epidemic) | 5 μg | 160 μg | 83% vs 0% (S) |
| | | CDC 13695#7 | Canada 2005 | 5 μg | 20 μg | 100% vs 8% (S) |
| | | | (epidemic) | | | |
| | | SP041 | US 2011 | 5 μg | 160 μg | 58% vs 0% (S) |
| IV | 023 | NK91 | France 2010 | 5 μg | 160 μg | 100% vs 0% (S) |
| A+B+ CDT+ | | | | | | |
| V | 078 | BAA-1875 | Hall O'Toole | 5 μg | 160 μg | 100% vs 0% (S) |
| A+B+ CDT+ | | | | | | |
| VIII | 017 | ATCC43598 | ATCC, Belguim, | 5 μg | 160 μg | 100% vs 44% (S) |
| A−B+ CDT− | | | ~1980's | | | |

This data demonstrates the C. difficile Toxoid Vaccine can confer broad protection against clinically relevant strains representative of the five most prevalent variant strains circulating worldwide in the hamster challenge model. The C. difficile Toxoid Vaccine induces broad protection in vivo against challenge with Toxinotype/PCR-ribotype 0/012, III/027, IV/023, V/078 and VIII/017 prototype strains, including those expressing binary toxin such as Toxinotypes/PCR-ribotypes III/027, IV/023 and V/078.

While certain embodiments have been described in terms of the preferred embodiments, it is understood that variations and modifications will occur to those skilled in the art. Therefore, it is intended that the appended claims cover all such equivalent variations that come within the scope of the following claims.

### REFERENCES

1. Khanafer N, Barbut F, Eckert C, Perraud M, Demont C, et al. (2016) Factors predictive of severe Clostridium difficile infection depend on the definition used. Anaerobe 37: 43-48.
2. Buckley A M, Spencer J, Maclellan L M, Candlish D, Irvine J J, et al. (2013) Susceptibility of hamsters to Clostridium difficile isolates of differing toxinotype. PLoS One 8: e64121.
3. Sambol S P, Tang J K, Merrigan M M, Johnson S, Gerding D N (2001) Infection of hamsters with epidemiologically important strains of Clostridium difficile. J Infect Dis 183: 1760-1766.

What is claimed is:

1. A method for immunizing a host against C. difficile strains expressing C. difficile binary toxin (CDT) and C. difficile strains not expressing CDT, the method comprising administering to the host an immunogenic composition comprising purified C. difficile Toxin A and purified C. difficile Toxin B inactivated by incubation with formaldehyde (w/v) at about 15-32° C. for about two to about 21 days wherein Toxin A is inactivated with 0.15%-0.5% formaldehyde (w/v) and Toxin B is inactivated with 0.15%-0.8% formaldehyde (w/v).

2. The method of claim 1 wherein the purified C. difficile Toxin A and purified C. difficile Toxin B are derived from a C. difficile strain that does not express C. difficile binary toxin (CDT).

3. The method of claim 2 wherein the purified C. difficile Toxin A and purified C. difficile Toxin B are derived from C. difficile strain VP110463/ATCC43255.

4. A method for inducing antibodies in a host, the antibodies having specificity for one or more C. difficile strains expressing C. difficile binary toxin (CDT), the method comprising administering to the host a composition comprising inactivated purified C. difficile Toxin A and inactivated purified C. difficile Toxin B derived from a C. difficile strain that does not express CDT.

5. The method of claim 4 wherein the antibodies are neutralizing as determined by a toxin neutralizing assay.

6. The method of claim 5 wherein the antibodies exhibit a relative efficacy (RE) of at least 5.4.

7. The method of any one of claims 4-6 wherein the antibodies neutralize toxin A and/or toxin B produced by a C. difficile strain having a toxinotype selected from the group consisting of 0, 1, III, IV, V, VI, VII, VIII, IX, and XII.

8. The method of claim 7 wherein:
the toxinotype 0 strain has the PCR-ribotype selected from the group consisting of 001, 002, 012, 014, 020, 014/020, 014/020/077, 106, 018, and 053;
the toxinotype III strain has the PCR-ribotype 027 or 075;
the toxinotype IV strain has the PCR-ribotype 023;
the toxinotype V strain has the PCR-ribotype selected from the group consisting of 078, 079, 122, 126, and 078/126;
the toxinotype VI strain has the PCR-ribotype 127 or 66-2;
the toxinotype VII strain has the PCR-ribotype 66-2;
the toxinotype VIII strain has the PCR-ribotype 017;
the toxinotype IX strain has the PCR-ribotype 019;
the toxinotype XIa strain has the PCR-ribotype 642;
the toxinotype XII strain has the PCR-ribotype 056.

9. The method of any one of claims 4-6 wherein the antibodies neutralize toxin A and/or toxin B produced by a C. difficile strain having PCR-ribotype 046 or 369.

10. The method of claim 7 wherein the antibodies neutralize toxin A and/or toxin B produced by a C. difficile strain having a toxinotype selected from the group consisting of 0, III, IV, V, and VIII.

11. The method of claim 7 wherein the antibodies neutralize toxin A and/or toxin B produced by C. difficile strains toxinotype 0, III, IV, V, and VIII.

12. The method of claim 10 or 11 wherein the toxinotype 0 strain has the PCR-ribotype 012, the toxinotype III strain has the PCR-ribotype 027, the toxinotype IV strain has the PCR-ribotype 023, the toxinotype V strain has the PCR-ribotype 078, and the toxinotype VIII strain has the PCR-ribotype 017.

13. The method of claim 7 wherein the antibodies neutralize toxin A and/or toxin B produced a C. difficile strains of toxinotype III, IV and V.

14. The method of claim 13 wherein the toxinotype III strain has the PCR-ribotype 027, the toxinotype IV strain has the PCR-ribotype 023, and the toxinotype V strain has the PCR-ribotype 078.

15. A method for immunizing and/or vaccinating a host against one or more C. difficile strains expressing C. difficile binary toxin (CDT), the method comprising administering to the host a composition comprising inactivated purified C. difficile Toxin A and inactivated purified C. difficile Toxin B derived from a C. difficile strain that does not express CDT.

16. The method of claim 15 wherein the host is immunized and/or vaccinated, respectively, against one or more C. difficile strains having a toxinotype selected from the group consisting of 0, III, IV, V and/or VIII.

17. The method of claim 15 or 16 wherein the host is immunized and/or vaccinated, respectively, against one or more C. difficile strains having a toxinotype selected from the group consisting of III, IV and V.

18. The method of claim 15 or 16 wherein the host is immunized and/or vaccinated, respectively, against one or more C. difficile strains having the toxinotypes III, IV and V.

19. The method of claim 18 wherein the toxinotype III strain has the PCR-ribotype 027, the toxinotype IV strain has the PCR-ribotype 023, and the toxinotype V strain has the PCR-ribotype 078.

20. The method of any one of claims 15-19 wherein significant protection against disease and death caused by C. difficile is provided to the host.

21. The method of claim 20 wherein protection is determined using the Golden Syrian hamster model.

22. The method of any one of claim 15-21 wherein the composition is administered to the host at least three times.

23. The method of claim 22 wherein the composition is administered via the intramuscular route.

24. The method of claim 22 or 23 wherein the composition is administered three times with two weeks between administrations.

25. The method of any one of claims 21-24 wherein the survival rate for a group of hamsters is about 58% to about 100%.

26. The method of any one of claims 21-25 wherein protection is statistically significant as determined by the Kaplan-Meier method with log-rank test and/or the bilateral Fisher exact test.

27. The method of claim 26 wherein, for a group of hamsters:
p=0.0001 with the Kaplan Meier log-rank test and p=0.0004 with the bilateral Fisher exact test;
p<0.001 with the Kaplan Meier log-rank test and p-value=0.005 with the bilateral Fisher exact test;
p-values ≤0.0001 with both the Kaplan Meier log-rank test and the bilateral Fisher exact test; and/or
p-value=0.0020 with the Kaplan Meier log-rank test and p=0.0046 with the bilateral Fisher exact test.

28. The method of any one of claims 1-27 wherein the composition does not include CDT or a subunit thereof.

29. The method of any one of claims 3-28 wherein the C. difficile Toxin A and Toxin B are derived from C. difficile Toxinotype 0.

30. The method of any one of claim 29 wherein the purified C. difficile Toxin A and purified C. difficile Toxin B are derived from C. difficile strain VPI10463/ATCC43255.

31. The method of any one of claims 3-30 wherein the Toxin A and Toxin B are inactivated by incubation with formaldehyde at about 15-32° C. for about two to about 21 days and wherein Toxin A is inactivated with 0.15%-0.5% formaldehyde (w/v) and Toxin B is inactivated with 0.15%-0.8% formaldehyde (w/v).

32. The method of any one of claims 1-31 wherein the composition comprises about 0.001% to 0.020% formaldehyde.

33. The method of claim 32 wherein the composition comprises about 0.004% formaldehyde.

34. The method of claim 32 wherein the composition comprises 0.008% formaldehyde.

35. The method of claim 32 wherein the composition comprises about 0.016% formaldehyde.

36. The method of any one of claims 1-35 wherein the Toxoid A and the Toxoid B are present in the composition in a A:B ratio of 5:1 to 1:5.

37. The method of claim 1-35 wherein the Toxoid A and the Toxoid B are present in the composition in a ratio of A:B of 3:1 or 3:2.

38. The method of any one of claims 1-37 wherein the composition is freeze dried, spray dried, or foam dried.

39. The method of any one of claims 1-37 wherein the composition is in liquid form.

40. The method of any one of claims 1-39, the composition further comprising one or more pharmaceutically acceptable excipients.

41. The method of claim 40 wherein the composition comprises a citrate, phosphate, glycine, carbonate, or bicarbonate buffer, or a pH-controlled aqueous solution.

42. The method of claim 40 or 41 further comprising a sugar, or sugar alcohol.

43. The method of any one of claims 40-42, the composition comprising sucrose and citrate.

44. The method of any one of claims 1 to 43, wherein the composition further comprises an adjuvant.

45. The method of claim 44 wherein the adjuvant comprises aluminum.

46. The method of claim 45 wherein the adjuvant comprises aluminum phosphate or aluminum hydroxide.

47. The method of claim 46 wherein the adjuvant comprises aluminum hydroxide.

48. The method of claim 47 wherein the composition comprises from about 20 μg to about 160 μg aluminum hydroxide.
